(19) United States
(12) Patent Application Publication
(10) Pub. No.: US 2024/0390613 A1
(43) Pub. Date: Nov. 28, 2024

(54) VALVED HOLDING CHAMBER FOR USE WITH DRY POWDER INHALER

(71) Applicant: Trudell Medical International Inc., London (CA)

(72) Inventors: Stephen Costella, London (CA); Matthew Gravett, London (CA); Sean Alexander Marshall, London (CA); Mark Nagel, Mt. Brydes (CA); Britton Sprickerhoff, London (CA); Jason Suggett, London (CA); Heather Young, London (CA)

(73) Assignee: Trudell Medical International Inc., London (CA)

(21) Appl. No.: 18/674,154

(22) Filed: May 24, 2024

Related U.S. Application Data
(60) Provisional application No. 63/468,922, filed on May 25, 2023.

(51) Int. Cl. A61M 15/00

(52) U.S. Cl. CPC A61M 15/0088; A61M 15/0016; A61M 2202/064; A61M 2205/0216; A61M 2205/073

(57) ABSTRACT

A valved holding chamber includes a holding chamber adapted to contain a substance and having an input end adapted to receive a dry powder inhaler, an output end and an interior volume defined between the input end and output end. An inhalation valve is disposed at the output end of the holding chamber and is moveable to an open position in response to an inhalation flow through the interior space. A vacuum generator, expandable volume and/or positive pressure may be used to draw medication from the dry powder inhaler into the holding chamber. Methods of using and assembling the system are also provided.

This application claims the benefit of U.S. Provisional Application Ser. No. 63/468,922, filed May 25, 2023, and entitled “Valved Holding Chamber For Use With Dry Powder Inhaler,” the entire disclosure of which is hereby incorporated herein by reference.

### TECHNICAL FIELD

The present invention relates generally to an aerosol delivery device, such as a valved holding chamber, which is configured for use with a dry powder inhaler, together with methods of delivering aerosol medicament and methods of assembling the delivery device.

### BACKGROUND

Dry powder inhalers (DPI) are known and available for aerosol medication delivery and may have a variety of shapes and sizes. In use, medicament is delivered during inspiration. Air flow through the DPI draws the medicament out of the DPI and causes the active pharmaceutical ingredient to separate from a carrier material (e.g. lactose), a process known as deagglomeration. For effective deagglomeration, the inspiratory flow must exceed a minimum threshold through the DPI (e.g., between 30-60 lpm). In addition, an abrupt rise in the flow is typically desired. The need for a powerful inspiration to actuate the DPI and create effective deagglomeration may limit the use of DPIs for certain users, such as children and adults who are unable to perform such inspiratory maneuvers. In these populations, a pressurized Metered Dose Inhaler (pMDI) or Soft Mist Inhaler (SMI) may be used as aerosol delivery devices, alone or in combination with a spacer or valved holding chamber (VHC).

VHCs are helpful for populations where inspiration and pMDI/SMI actuation are uncoordinated. The VHC slows the emitted aerosol and maintains a suspension of the aerosol long enough for users to receive an adequate dose while breathing tidally. The need for powerful inspiration associated with the DPI in combination with the suspension of the aerosol in the VHC as it is accessed by a user has led to difficulties in mating the two devices.

### SUMMARY

In one aspect, one embodiment of a valved holding chamber includes a holding chamber adapted to contain a substance and having an input end adapted to receive a dry powder inhaler, an output end and an interior volume defined between the input end and output end. An inhalation valve is disposed at the output end of the holding chamber and is moveable to an open position in response to an inhalation flow through the interior space. A vacuum generator is in fluid communication with the interior space, wherein the vacuum generator is configured to create a vacuum in the interior space.

In another aspect, one embodiment of a valved holding chamber includes a holding chamber adapted to contain a substance and having an input end adapted to receive a dry powder inhaler, an output end and an expandable interior volume defined between the input end and output end. An inhalation valve is disposed at the output end of the holding chamber and is moveable to an open position in response to an inhalation flow through the interior space. An expander is coupled to the holding chamber, wherein the expander is moveable from a first configuration, wherein the expandable interior volume comprises a first interior volume, and a second configuration, wherein the expandable interior volume comprises a second interior volume, wherein the second interior volume is greater than the first interior volume. In operation, the expander is moved to the second configuration to actuate the DPI.

In yet another aspect, one embodiment of a valved holding chamber includes a holding chamber adapted to contain a substance and having an input end adapted to receive a dry powder inhaler, an output end and an interior volume defined between the input end and output end. An inhalation valve is disposed at the output end of the holding chamber and is moveable to an open position in response to an inhalation flow through the interior space. A positive pressure source communicates with the input end. In various embodiments, the positive pressure source may communicate with an inlet of the DPI, or may be disposed between the outlet of the DPI and the input end of the VHC.

In another aspect, one embodiment of a method of delivering an aerosolized medication includes operably connecting a dry powder inhaler to an input end of a holding chamber, creating a vacuum in an interior space of a holding chamber, drawing medication from the dry powder inhaler with the vacuum into the interior space of the holding chamber, and inhaling the medication through an output end of the holding chamber. In one embodiment, the aerosolized medication passes through an inhalation valve at the output end of the holding chamber during inhalation.

In another aspect, one embodiment of a method of delivering an aerosolized medication includes operably connecting a dry powder inhaler to an input end of a holding chamber, expanding an interior volume of the holding chamber and thereby drawing medication from the dry powder inhaler into the interior volume of the holding chamber, and inhaling the medication through an output end of the holding chamber. In one embodiment, the aerosolized medication passes through an inhalation valve at the output end of the holding chamber during inhalation.

In another aspect, one embodiment of a method of delivering an aerosolized medication includes operably connecting a dry powder inhaler to an input end of a holding chamber, applying a positive pressure to the holding chamber and thereby entraining medication through the dry powder inhaler and inhaling the medication through an output end of the holding chamber. In one embodiment, the aerosolized medication passes through an inhalation valve at the output end of the holding chamber during inhalation.

The various aspects and embodiments provide significant advantages over other medication delivery systems and methods. For example, in the same way VHCs address coordination issues with pMDIs and SMIs, it may be desirable to combine a DPI with a VHC if an adequate flow rate is generated to facilitate deagglomeration. The resulting DPI aerosol plume may then be captured in the VHC and made available for tidal inhalation. In the various aspects and embodiments, the holding chambers create a sufficient vacuum, change in volume and/or positive pressure sufficient to deagglomerate the dry powder medication, such that it may be aerosolized and suspended in the valved holding chamber. Whereinafter, the aerosolized medication may be inhaled by the user, for example through tidal breathing.

The foregoing paragraphs have been provided by way of general introduction, and are not intended to limit the scope of the following claims. The various preferred embodiments, together with further advantages, will be best understood by reference to the following detailed description taken in conjunction with the accompanying drawings.

### BRIEF DESCRIPTION OF THE DRAWINGS

FIG. 1 is a side view of one embodiment of a dry powder inhaler (DPI).

FIG. 2 is a diagram showing various actuation mechanisms for the VHC.

FIG. 3 is a side schematic view of one embodiment of a DPI coupled to a VHC.

FIG. 4A is a side schematic view of another embodiment of a DPI coupled to a VHC with the vacuum generator in a pre-actuated state.

FIG. 4B is a side schematic view of the embodiment of the DPI coupled to the VHC shown in FIG. 4A with the vacuum generator in an actuated state.

FIG. 5 is a side schematic view of another embodiment of a DPI coupled to a VHC.

FIG. 6 is a side schematic view of another embodiment of a DPI coupled to a VHC.

FIG. 7 is a partial side cross-sectional view of another embodiment of a DPI coupled to a VHC.

FIG. 8 is a graph of an initial pressure P1 v VHC volume.

FIG. 9 is a schematic illustration of one embodiment of a delivery system including a constant flow resistor.

FIG. 10 is a schematic illustration of another embodiment of a delivery system including a constant flow resistor.

FIG. 11A is a side schematic view of another embodiment of a DPI coupled to a VHC with the vacuum generator in a pre-actuated state.

FIG. 11B is a side schematic view of the embodiment of the DPI coupled to the VHC shown in FIG. 11A with the vacuum generator in an actuated state.

FIG. 12A is a side schematic view of another embodiment of a DPI coupled to a VHC with an expander in a first position.

FIG. 12B is a side schematic view of the embodiment of the DPI coupled to the VHC shown in FIG. 12A with the expander in a second position.

FIG. 13 is a side schematic view of another embodiment of a DPI coupled to a VHC with an expander successively moved from a first position to a second position and back to a first position.

FIG. 14A is a side schematic view of another embodiment of a DPI coupled to a VHC with an expander in a first position.

FIG. 14B is a side schematic view of the embodiment of the DPI coupled to the VHC shown in FIG. 14A with the expander in a second position.

FIG. 15A is a side schematic view of another embodiment of a DPI coupled to a VHC with an expander in a first position.

FIG. 15B is a side schematic view of the embodiment of the DPI coupled to the VHC shown in FIG. 15A with the expander in a second position.

FIG. 16 is a side schematic view of another embodiment of a DPI coupled to a VHC.

FIG. 17 is a side schematic view of one embodiment of a DPI coupled to a VHC and including a positive pressure source.

FIG. 18 is a side schematic view of another embodiment of a DPI coupled to a VHC and including a positive pressure source.

FIG. 19 is a side schematic view of another embodiment of a DPI coupled to a VHC and including a positive pressure source.

FIG. 20 is a side schematic view of another embodiment of a DPI coupled to a VHC and including a positive pressure source.

FIG. 21 is a side schematic view of one embodiment of a DPI coupled to a backpiece with a coupler.

FIG. 22 is a side schematic view of another embodiment of a DPI coupled to a backpiece with a coupler.

FIG. 23 is one embodiment of a backpiece with a universal DPI coupler.

FIG. 24A is another embodiment of a backpiece with a retractable universal DPI coupler.

FIG. 24B is another embodiment of a backpiece with a retractable universal DPI coupler.

FIG. 25 is another embodiment of a backpiece with a universal DPI coupler.

FIG. 26 is another embodiment of a backpiece with a universal DPI coupler.

FIG. 27 is another embodiment of a backpiece with a universal DPI coupler.

FIG. 28 is a side schematic view of an adapter for coupling a DPI to a VHC.

FIG. 29 is a side schematic view of a VHC with a backpiece having an integrated DPI coupler.

FIG. 30 is a side schematic view of a VHC having a backpiece expander.

FIG. 31 is a side schematic view of a VHC having an integrated DPI.

FIG. 32 is a side schematic view of a VHC having a DPI carrier.

FIG. 33 is an exploded, perspective view of another embodiment of a DPI coupled to a VHC.

FIG. 34 is a front, perspective view of the DPI and VHC shown in FIG. 33 in an assembled state.

FIG. 35 is a cross-sectional view of the DPI and VHC shown in FIG. 34 in a collapsed configuration as inhalation is initiated.

FIG. 36 is a cross-sectional view of the DPI and VHC shown in FIG. 34 in an extended configuration during inhalation.

FIG. 37 is a cross-sectional view of the DPI and VHC shown in FIG. 34 in an extended configuration during exhalation.

FIG. 38 is a top view of the DPI and VHC shown in FIG. 34 in a collapsed configuration.

FIG. 39 is a top view of the DPI and VHC shown in FIG. 34 in a collapsed configuration.

FIG. 40 is a perspective view of the VHC shown in FIG. 34 with a metered dose inhaler (MDI) coupled thereto.

FIG. 41 is a top view of an alternative embodiment of a VHC in a collapsed configuration.

FIG. 42 is a side view of the VHC shown in FIG. 41 in a collapsed configuration.

FIG. 43 is a perspective view of the VHC shown in FIG. 41 in a collapsed configuration.

FIG. 44 is an cross-sectional view of a DPI coupled to a VHC.

FIG. 45 is a rear, partial view of a backpiece with an inhalation valve in an open position during inhalation.

FIG. 46 is a front view of an actuator.

FIG. 47 is a cross-sectional view of the actuator taken along line 47-47 in FIG. 46.

FIG. 48 is a top view of the actuator shown in FIG. 46.

FIG. 49 is a side view a collapsible wall in a collapsed configuration.

FIG. 50 is a side view of the collapsible wall in an extended configuration.

FIG. 51 is a top view of one embodiment of a VHC.

FIG. 51A is a perspective view of the VHC shown in FIG. 51.

FIG. 52 is a front view of the VHC shown in FIG. 51.

FIG. 53 is a side view of one embodiment of a backpiece.

FIG. 54 is a rear view of the backpiece shown in FIG. 53.

FIG. 55 is a cross-sectional view of the backpiece take along line 55-55 in FIG. 53.

FIG. 56 is a front view of the backpiece shown in FIG. 53.

FIG. 57 is a side view of another embodiment of a backpiece.

FIG. 58 is a rear view of the backpiece shown in FIG. 57.

FIG. 59 is a cross-sectional view of the backpiece take along line 59-59 in FIG. 57.

FIG. 60 is a front view of the backpiece shown in FIG. 57.

FIG. 61 is an alternative embodiment of a holding chamber with a pair of collapsible walls.

FIG. 62 discloses a plan view of a holding chamber configured with one embodiment of a user interface adapter.

FIG. 63 discloses a plan view of a holding chamber configured with another embodiment of a user interface adapter.

FIG. 64 discloses a plan view of a holding chamber configured with another embodiment of a user interface adapter.

FIG. 65 discloses a plan view of a holding chamber configured with another embodiment of a user interface adapter.

FIG. 66 discloses a plan view of a holding chamber configured with another embodiment of a user interface adapter.

FIG. 67 discloses a plan view of a holding chamber configured with another embodiment of a user interface adapter.

FIG. 68 discloses a plan view of a holding chamber configured with another embodiment of a user interface adapter.

FIG. 69 discloses a plan view of a holding chamber configured with another embodiment of a user interface adapter.

### DETAILED DESCRIPTION OF THE DRAWINGS AND PRESENTLY PREFERRED EMBODIMENTS

It should be understood that the term “plurality,” as used herein, means two or more. The terms “longitudinal” as used herein means of or relating to length or the longitudinal direction 2, for example between the opposite ends of the holding chamber. The terms “lateral” and “transverse” as used herein, means situated on, directed toward or running from side to side (front and back of a worksurface), and refers to a lateral direction 4 orthogonal to the longitudinal direction. The term “direction” corresponds to an axis or line, rather than a vector. The term “coupled” means connected to or engaged with, whether directly or indirectly, for example with an intervening member, and does not require the engagement to be fixed or permanent, although it may be fixed or permanent (or integral), and includes both mechanical and electrical connection. The terms “first,” “second,” and so on, as used herein are not meant to be assigned to a particular component so designated, but rather are simply referring to such components in the numerical order as addressed, meaning that a component designated as “first” may later be a “second” such component, depending on the order in which it is referred. For example, a “first” side may be later referred to as a “second” side depending on the order in which they are referred. It should also be understood that designation of “first” and “second” does not necessarily mean that the two features, components or values so designated are different, meaning for example a first side may be the same as a second side, with each simply being applicable to separate but identical components. As used herein: the term “substance” includes, but is not limited to, any substance that has a therapeutic benefit, including, without limitation, any medication; the terms “user” and “patient” includes humans and animals; and the term “aerosol delivery devices or system(s)” includes dry powder inhalers (DPIs), add-on devices, such as VHCs, and devices including a chamber housing and integrated actuator suited for a DPI.

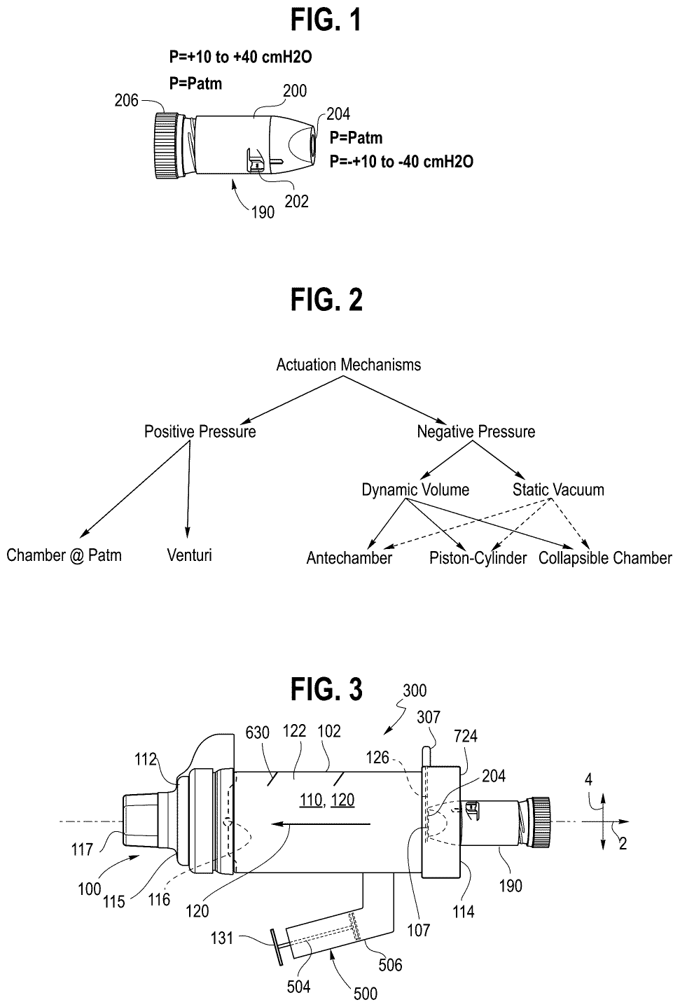

FIG. 1 shows a dry powder inhaler (DPI) 190. The DPI includes a chamber housing 200 defining an interior space for holding the medication, which may take the form of a capsule 157 or predetermined doses disposed in one or more recesses or holding receptacles. The DPI includes an inlet 202, through which air is drawn and entrains the medicament thereby removing the medicament from the capsule or receptacle. The DPI also includes an outlet 204 through which the medicament passes, configured for example as a mouthpiece. Various dry powder inhalers are disclosed for example and without limitation in U.S. Pat. Nos. 4,627,432 and 6,116,239, the entire disclosures of which are incorporated herein by reference.

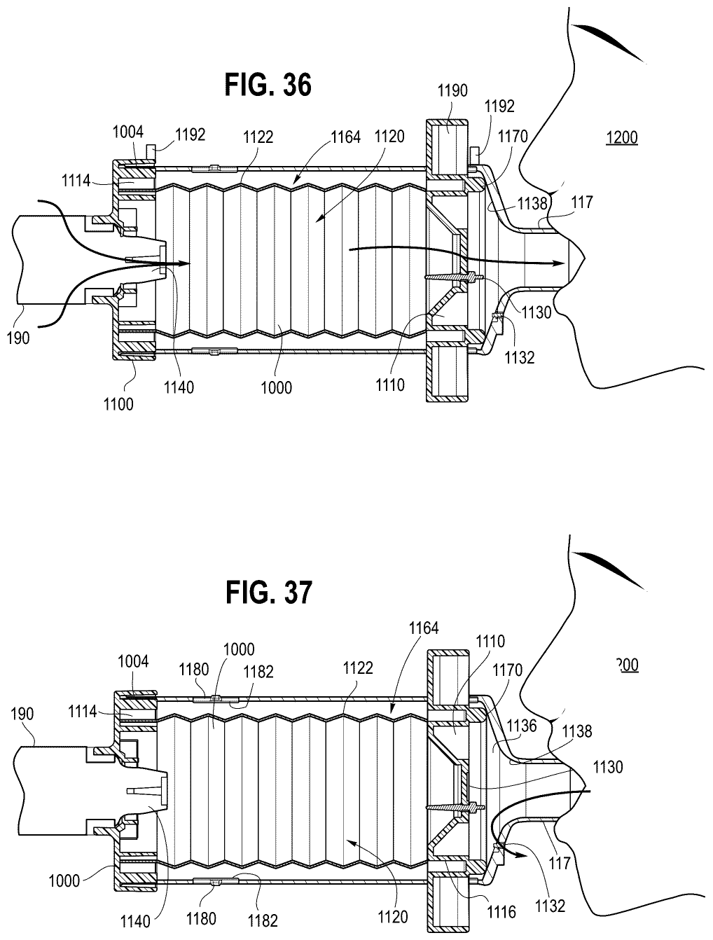

In operation, the patient activates the chamber housing 200, for example by rotating an actuator 206, to provide access to the dry powder. A user 1200 is shown as interfacing with a device in FIGS. 36, 37 and 64. It should be understood that the user may similarly interface with the other chamber housing and device embodiments shown in the other Figures, with the depiction of the user being omitted for the sake of simplicity and clarity. The user then places their mouth on the mouthpiece and inhales through the outlet 204, which creates a flow path entraining the dry powder substance. The aerosolized substance travels through the outlet 204 to reach the patient via the mouthpiece. The DPI may require some procedure, e.g., rotation of the actuator, to allow a measured dose of powder to be readied for the patient to take, for example by puncturing a capsule or exposing a receptacle. The medication is commonly held either in a capsule for manual loading or in a powder form disposed in a receptacle inside the inhaler. Once loaded or actuated, the user inhales through the outlet 204. The present embodiments are not limited to the treatment of human patients. For example, it is contemplated that the DPI may be incorporated into a delivery system for administering medication to animals, including for example and without limitation equines, cats, dogs, and other animals.

Referring to FIGS. 3-7 and 11A-20, a valved holding chamber (VHC) 100 includes a chamber housing 102, or holding chamber, adapted to contain a substance and having an input end 107 adapted to receive the dry powder inhaler 190, and be in fluid communication with the outlet 204. An output end 112 is in flow communication with a user interface 117, which may be configured as a mouthpiece, mask (nasal or oral/nasal), tube, or other suitable user interface, which is in fluid communication with the output end 112. The holding chamber has an interior space 120 and defines an interior volume 110 defined between the input end 107 and output end 112. An inhalation valve 116 is disposed at the output end 112 of the holding chamber and is moveable to an open position in response to an inhalation flow 121 through the interior space. The chamber housing 108 may have a generally cylindrical cross-sectional shape that defines the interior volume 110 of space for receipt therein of aerosolized medication from the DPI 190. The output end may be configured with an opening that is in fluid communication with the interior volume 110 or interior space 120 of the chamber housing 108. The inhalation valve 116 may be disposed over the opening to provide for one-way flow through the opening during inhalation but prevents back flow into the interior volume 110 during exhalation. An exhalation valve 115 may be provided to allow for one-way flow to the ambient environment surrounding the VHC 100 during exhalation, but prevents air entrainment through an exhalation opening during inhalation. The input end 107 of the chamber housing 108 may include a detachable and flexible backpiece 114, or adapter, that defines or includes an inlet, configured in one embodiment as an opening suited to receive the mouthpiece portion of the DPI.

The VHC 100 may be used with the DPI 190 to permit user-friendly DPI actuation. An actuation mechanism 300 may be configured such that at the push of a button 307 (or other trigger), an adequate flow rate (e.g. 30-60 Lpm) is forced through the DPI 190 to facilitate deagglomeration. The resulting DPI aerosol plume may then be captured in the interior volume 110 of the VHC or spacer and made available for tidal inhalation. The DPI 190 will disperse medication in an effective manner when flows of 30-60 Lpm are drawn through the DPI 190. Alternatively, rapid application of a pressure gradient of 10-40 cmH2O will result in sufficient flow for deagglomeration, with the lower pressure in the gradient being applied at the DPI mouthpiece, or outlet 204, as shown in FIG. 1. The DPI mouthpiece is releasably coupled to the input end 107 of the VHC, as further disclosed below in connection with various embodiments, in an airtight manner, preferably with no alteration to the DPI or drug capsules contained or loaded therein. Referring to FIG. 2, the actuation mechanism 300 may be used to created a negative pressure, by way of a static vacuum or by creating a dynamic volume change, or by way of applying a positive pressure.

### Static Vacuum

In one embodiment, a vacuum generator 500 is in fluid communication with the interior space 120, wherein the vacuum generator is configured to create a vacuum in the interior space 120. In one embodiment, the vacuum generator 500 generates a static −10 to −40 cmH2O vacuum within the interior space 120. In response to the actuation of the actuation mechanism 300, the vacuum or negative pressure is applied rapidly to the DPI outlet 204, for example through the mouthpiece. The actuation mechanism 300 may include, and be actuated for example by actuating the trigger 307. Pressure within the VHC just before the trigger 307 is actuated may be −10 to −40 cmH2O.

In one embodiment, as shown in FIG. 3, the interior space 120 includes a main chamber 122 separated from an ante chamber 124 by a release valve 126. The vacuum generator 500 includes a retractable piston 504 movably supported by a cylinder 506. In one embodiment, the cylinder defines an auxiliary chamber fluidly connected with the main chamber 122. The vacuum generator 500 is in fluid communication with the main chamber 122 such that a vacuum is created in the main chamber 122, while the ante chamber 124 is in fluid communication with the input end 107 and DPI outlet 204. A vacuum of −10 to −40 cmH2O is generated when the piston 504 is retracted in the cylinder 506 by the user, for example by gripping and pulling on a handle 131. The antechamber 124 has a much small volume than the main chamber 122. In operation, the DPI 190 is positioned and coupled to the VHC such that outlet 204 is in fluid communication with the ante chamber 124, for example by inserting the mouthpiece through the backpiece 114 and into the ante chamber 124. The DPI inlet 202, i.e., air inlet(s), are positioned outside of both chambers such that the inlet(s) is exposed to the ambient environment or atmosphere. In the closed position, the movable release valve 126 fluidly isolates the antechamber 124 from the main chamber 122 and can maintain a pressure difference of at least −10 to −40 cmH2O across and between the chambers 122, 124 without leaking. The actuation mechanism 300, for example the trigger 307, may be actuated so to move the valve 126 from a closed position to an open position, whereinafter the main chamber 122 and antechamber 124 become fluidly connected and pressures rapidly reach an equilibrium, preferably a negative pressure of at least −10 to −40 cmH2O. The movable release valve 126 may be spring loaded such that at the push of the button 307, the valve 126 will move rapidly from the closed position to the open position. In operation, and before actuation, the piston 504 is in a fully depressed position and the movable valve 126 is in the closed position. To actuate the system, the user retracts the piston 504, or pulls the piston 504 away from the main chamber 122 in the cylinder 506 to generate the vacuum in the main chamber 122. When the user is ready to inhale, the user, or caregiver, actuates the trigger 307, for example by pushing the button, which moves the release valve 126 from the closed to open position. The −10 to −40 cmH2O pressure in the main chamber 122 is permitted to rapidly equilibrate with the pressure of the antechamber 124, exposing the DPI mouthpiece, and outlet 204, to the negative pressure, which dispenses and deagglomerates the medication in the DPI. The medication is then held in suspension in the interior space 120 for the user to inhale tidally, for example by inhaling through the output end 112 and the inhalation valve 116. The valve 126 and piston 504 may then be reset to permit additional DPI actuations.

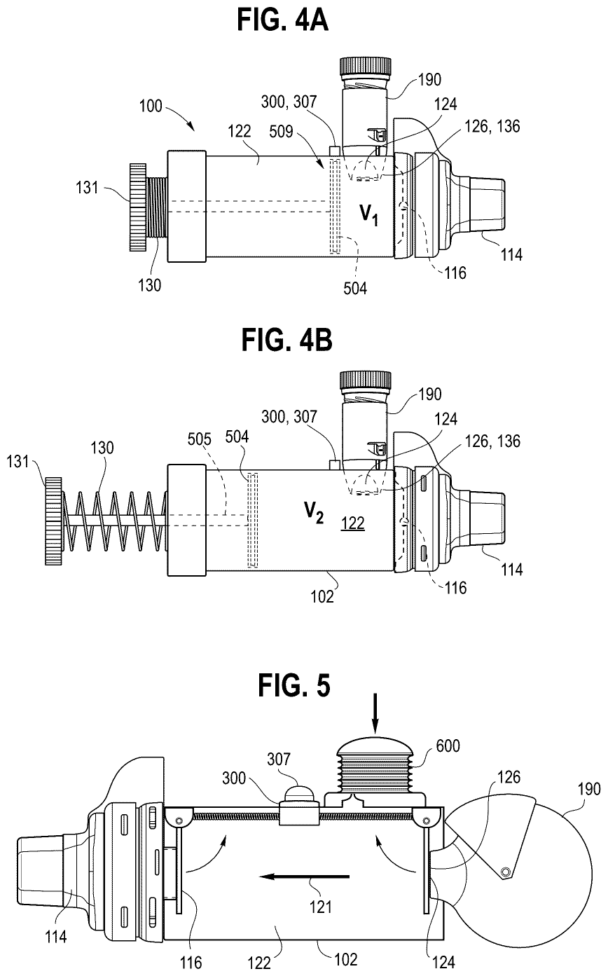

In an alternative embodiment, shown in FIGS. 4A and 4B, the retractable piston 504 is disposed in the main chamber 122. The piston may include the grippable portion 131, or handle, exposed to the ambient environment at one end of the holding chamber 100. A return spring 130 biases the piston 504, for example by engaging the grippable portion 131, to an at-rest position. The piston 504, or vacuum generator, may be retracted against the force of the spring 130 in the main chamber 122 to create the vacuum. In this embodiment, the DPI 190 is in fluid communication with the ante chamber 124 near the output end 112. The actuation mechanism 300, including for example a trigger 307, may be actuated to open as valve 126 to expose the ante chamber 124 to the main chamber 122.

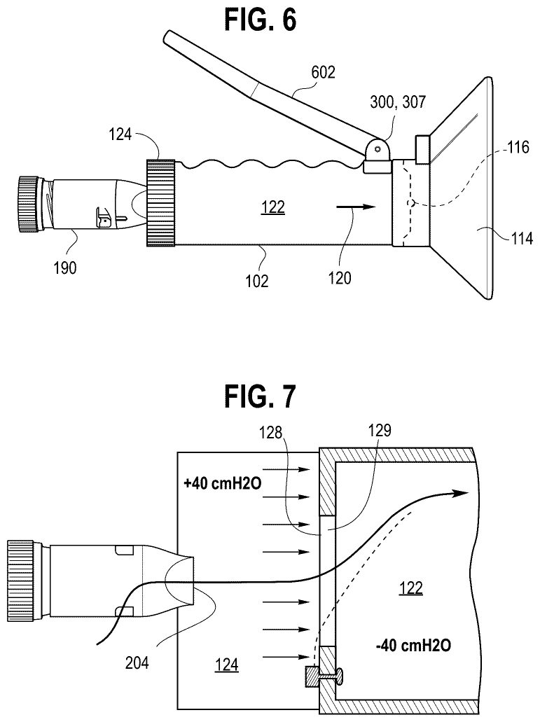

In an alternative embodiment, shown in FIGS. 5 and 6, the piston-cylinder vacuum generator may be replaced by an electric pump or hand pump 600, 602 to generate the desired vacuum. In one embodiment, the trigger 307 may actuate and simultaneously move both the release valve 126 and inhalation valve 116. In another embodiment, the user's tidal inspiratory efforts may be used to generate the required vacuum, possibly over the course of multiple breaths.

In an alternative embodiment, the push-button trigger 307 may be replaced by a pressure trigger mechanism which automatically moves the valve 126 from the closed to open position when a predetermined negative pressure, e.g., −10 to −40 cmH2O, is reached in the main chamber 122. For example, and without limitation, and referring to FIG. 7, a flexible, quick release valve 128 covers an opening 129 that separates the main chamber 122 from the antechamber 124. As the user inhales, or another vacuum generating mechanism acts, the pressure in the main chamber 122 drops until a predetermined trigger pressure is reached (e.g., −10 to −40 cmH2O), whereinafter the valve 128 is opened by pulling a portion of the valve 128 through the opening 129 between the antechamber 124 and main chamber 122, fluidly connecting the two chambers. The movement of the valve 128 permits tidal inhalation of the suspended aerosol from the holding chamber through the inhalation valve 116. The user then resets the flexible valve 128 prior to the next DPI dose being administered. To minimize impaction in this design, the DPI mouthpiece, and outlet 204, may be positioned as close as possible to the valve 128 in the region where the valve bursts through its opening 129.

In each of the embodiments described above, the volume of air pulled through the DPI 190 needs to be sufficient to pull the entire drug dose out of the DPI 190. Depending on the configuration of the DPI, the volume is preferably between 400 mL-1,000 mL. As the mass of air accumulates in the VHC, the pressure within the main chamber 122 of the VHC will increase towards atmospheric pressure according to the Ideal Gas Law. With this in mind, the volume of the main chamber 122 of the VHC is a factor in maintaining a sufficient vacuum while also permitting 400 mL-1,000 mL of fresh air to enter the chamber 122.

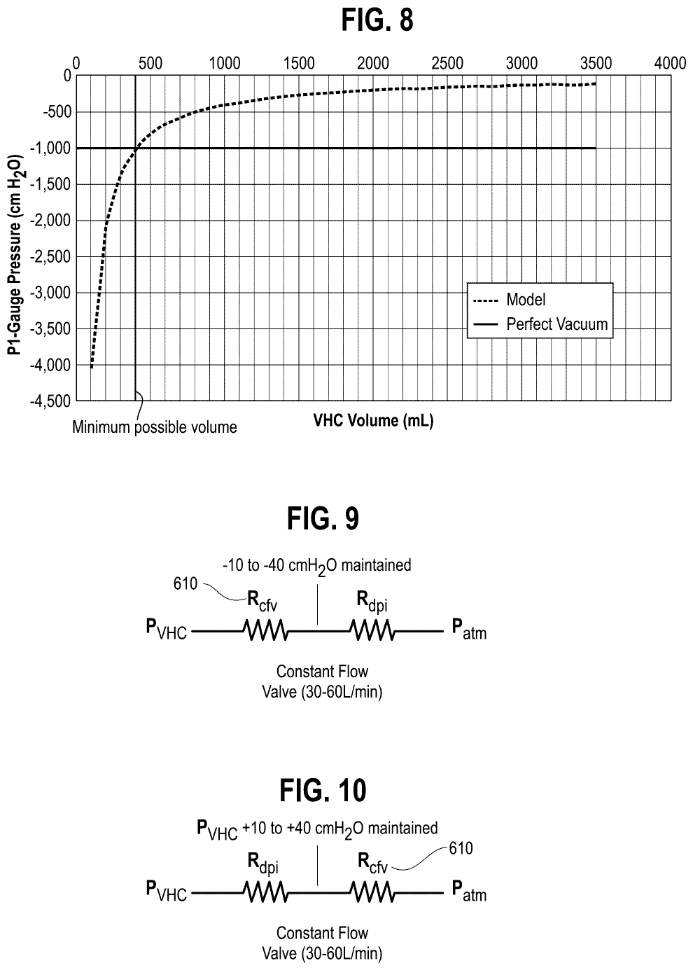

As shown in FIG. 8, a graphs of the minimum vacuum pressure v. VHC volume required to permit 400 mL of fresh air to enter the VHC before the pressure increases above −10 cmH2O is derived from Ideal Gas Law. According to this model, if only a −40 cmH2O vacuum is generated within the VHC main chamber 122, the VHC chamber volume would need to be about 13.3 L, which is an impractical volume for many users and portable applications. To decrease the size of the VHC, therefore, a larger magnitude vacuum pressure is needed. For a VHC whose chamber volume is 500 mL, for example, the vacuum source will need to be able to generate −821 cmH2O in the main chamber, as shown in FIG. 8. Other acceptable negative pressures associated with various volumes may be discerned from FIG. 8.

For the piston-cylinder embodiments of FIGS. 3-4B, the vacuum generated will be proportional to the change in volume but also inversely proportional to the initial volume of the VHC (from Boyle's Law): P₁V₁=P₂V₂. Since a fixed volume of the main chamber 122 is required to slow and suspend the aerosol, a fluidly connected, parallel piston-cylinder volume would need to be much larger to permit changes of volume that adequately accommodate this large V₁. Therefore, to keep the system compact, the embodiment of FIGS. 4A and 4B, which houses the piston within the main chamber volume, may provide certain advantages. Specifically, the change in volume is directly applied to the main chamber as shown in FIGS. 4A and B. Prior to retracting the piston, V1 is very small. Once fully retracted, the VHC volume V2 becomes 500 mL.

Large negative pressures may also lead to impaction. Larger pressure drops across the DPI 190 may result in higher flow rates and higher velocity/momentum of the drug particles. Significant momentum makes the particles more likely to impact and not reach the intended target region in the patient if forced to navigate abrupt direction changes. This presents a challenge when trying to make a VHC compact because generally, a larger space is required to adequately slow the aerosol. To slow the aerosol enough without making the size of the VHC impractical, a tangential introduction of the aerosol into the chamber along the chamber circumference may be beneficial. The diameter of the chamber needs to be large enough such that the aerosol plume can follow a contour without impacting on the sides.

Alternatively, and referring to FIG. 9, a constant flow resistor 610 may be incorporated between the DPI outlet 204 and the main chamber volume 110 in such a way as to not cause significant impaction losses. For example, in one embodiment, the resistor 610 may contain a flexible orifice or opening whose diameter may grow and shrink passively in response to the pressure gradient across the resistor opening such that a flow of 30-60 Lpm is always maintained. Just prior to initial actuation of the DPI 190, the pressure in the main chamber 122 may be −821 cmH2O, or lower. This resistor 610 will behave such that the pressure on the other side of the orifice is maintained at or near the −10 to −40 cmH2O, regardless of the main chamber 122 pressure. The resistor will permit the aerosol to enter the main chamber 122 chamber at lower speeds, reducing impaction. Alternatively, the constant flow resistor 610 may be positioned upstream of the DPI, as shown in FIG. 10. Referring to FIGS. 9 and 10, P_{VHC} represents the air pressure within the VHC and Pₐₜₘ represents atmospheric pressure. The 10 to 40 cmH2O is gauge pressure.

In another embodiment, auxiliary air inlets 630 may be incorporated into the VHC such that additional air flow can enter the holding chamber through paths other than through the DPI 190. These inlets 630 may be arranged such that the direction and velocity of airflow is opposite and comparable to that of the airflow coming through the DPI 190. This counterflowing air can serve to slow the aerosol plume coming through the DPI 190.

### Dynamic Volume

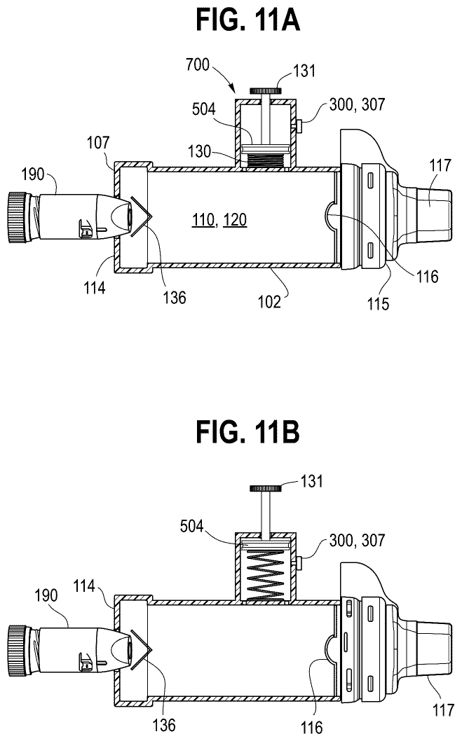

Referring to FIGS. 11A and B, the 30-60 Lpm flow rate may also be generated by rapidly increasing the chamber volume (or fluidly connected volume) with an expander 700 after an actuation mechanism 300, including for example a trigger 307, is applied. Pressure within the VHC just before the trigger is actuated is atmospheric (i.e. 0 cmH2O gauge), in contrast to the embodiments where a negative pressure is applied to the main chamber. After actuation, −10 to −40 cmH2O pressure is applied to the system to actuate the DPI 190.

The expander 700 is coupled to the holding chamber and is moveable from a first configuration, wherein the expandable interior volume is a first interior volume, and a second configuration, wherein the expandable interior volume is a second interior volume, wherein the second interior volume is greater than the first interior volume. The system shown in FIGS. 4A and 4B may also function as an expander, for example if the valve 126 is removed, or is replaced with a one-way valve 136 that opens in response to the pressure change created by the expander being actuated rather than in response to a trigger. The valve 136 functions merely as a one-way inhalation valve allowing flow through the valve during inhalation, but precludes any flow out through the DPI during exhalation or during reset of the expander 700. The valve 136 does not maintain any pressure differential during inhalation, but rather open to allow flow from the DPI. The expander 700 may be include the piston 504 disposed in the interior volume. The spring 130 biases the piston 504 from a first position, wherein the piston defines in part the first interior volume, to a second position, wherein the piston 504 defines in part the second interior volume. The actuator 300 is coupled to the piston 504 and/or spring 130, wherein the actuator, which may include the trigger 307, is configured to release the spring 130 so as to bias the piston 504 from the first position to the second position. In one embodiment, the piston 504 is disposed in the main chamber, although the piston may be disposed in an auxiliary chamber.

In one embodiment, shown in FIGS. 4A and 4B, the DPI 190 mouthpiece and outlet 204 is exposed directly to the main chamber volume 122 and is positioned near the front of the chamber, i.e. proximate or adjacent the inhalation valve 116 of the VHC. In this embodiment, the “main chamber volume” is defined as the volume between the inhalation valve and a movable piston 504. This piston 504 is free to translate along the longitudinal axis of the chamber, thereby permitting the chamber volume to change. Attached to this piston 504 is the expander spring 130, configured in one embodiment as a compression spring, although the spring may be configured as a tension, torsion, leaf or other type of spring. When the piston 504 is moved to a position such that chamber volume is minimized, the expander spring 130 is compressed (or loaded) and the piston 504 or guiding rod 505 engages with a detent 509, or catch, to lock it in position. This detent can be released at the push of the button (the “trigger”) 307 such that the compression spring 130 rapidly translates the piston 504 to the position where the chamber volume is maximized. The spring 130 is sized such that this translation occurs at such a velocity that 30-60 Lpm is pulled through the DPI 190 into the chamber. The dispensed, aerosolized drug is then held suspended, ready for tidal inhalation in the VHC. During reset, a one-way valve 126 prevents flow travelling into the DPI and out to atmosphere as this may eject medication from the DPI or cause damage to the DPI.

In an alternative embodiment, shown in FIGS. 11A and B, the expander 700 includes a piston 504 sliding within an auxiliary chamber fluidly connected with the main chamber. The auxiliary chamber may be approximately 400 mL in volume but adjustable depending on the DPI being used. The DPI 190 may be attached to the VHC in the usual MDI position via a backpiece 114. In this embodiment, the main chamber is free of any piston. The piston may be spring loaded with a spring 130, as previously discussed and shown in FIG. 11A, or may include a pull handle 131 that is rapidly pulled by the user to generate sufficient flows as shown in FIG. 11B. A one-way valve 136 prevents backflow through the DPI 190 during reset.

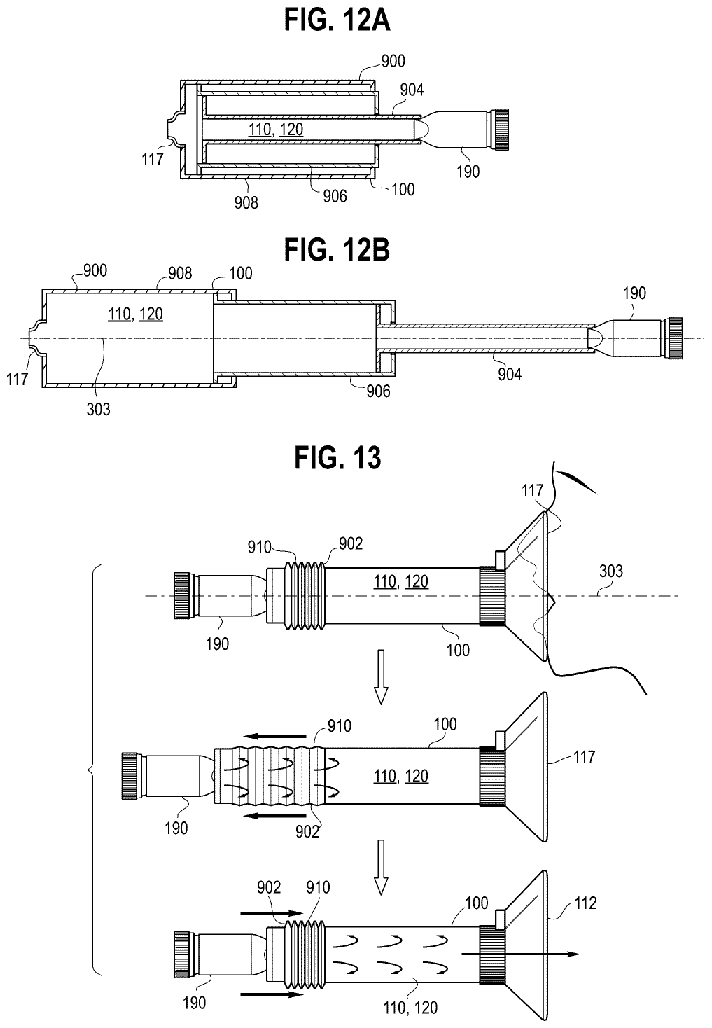

In other embodiments, shown in FIGS. 12A and B and 13, the main chamber volume is collapsible along the axis 303 of the chamber, with the chamber including a collapsible wall 900, 902. In one embodiment, the collapsible wall includes telescoping segments 904, 906, 908, which may be moved relative to each other to rapidly increase the volume as shown in FIGS. 12A and B. In another embodiment, the collapsible wall 902 includes circumferential pleats 910, which may be collapsed and expanded as shown in FIG. 13. In either embodiment, with the chamber collapsed and the DPI 190 attached, the user rapidly pulls the chamber in a longitudinal direction such that the chamber, or collapsible wall 900, 902, expands from its collapsed state to its expanded state. The rate of pull is such that 30-60 Lpm gets pulled through the DPI 190 and into the VHC 100. Alternatively, the expansion can be used to generate the −10 to −40 cmH2O in a main chamber, with a spring-release valve allowing for the vacuum to be applied to the DPI. The collapsible wall 900 902 allows the VHC to be made more compact and easy to carry when not in use.

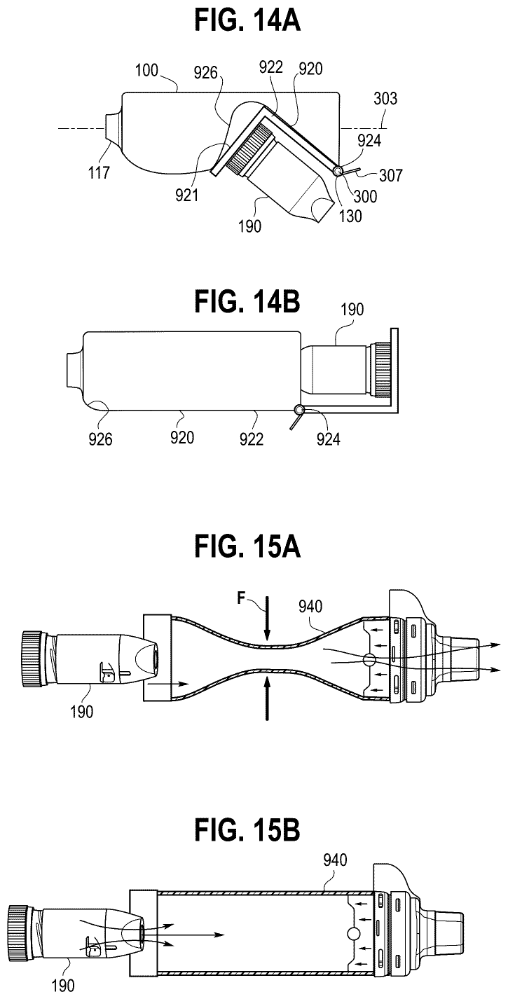

In an alternative embodiment, shown in FIGS. 14A and B, the chamber is not collapsible along the longitudinal axis 303. Rather, a collapsible wall 920 partially folds inwardly, with the collapsed volume sufficiently smaller than the expanded volume. The wall 920 may include a rigid portion 922 having one end pivotally or hingedly attached to a portion of the holding chamber 100 at a pivot axis 924 and a flexible portion 926 connecting another end of the rigid portion to the holding chamber 100. In various embodiments, the chamber may expand from a collapsed state to an expanded state by way of an actuation mechanism 300, for example at the push of a button 307 (the “trigger”). In these embodiments, the expanding force can be provided by a spring 130, or the expander force may be integrated into the circumferential pleats 910 such that the pleats define a spring.

In one embodiment, the actuation mechanism is the chamber itself, which may be the squeezed, with elastically deformable side walls 940 deformed to collapse the chamber and decrease the volume as shown in FIGS. 15A and B. The expander is defined by the deformable side walls 940, which return to their original shape when released. As the user squeezes the chamber, the chamber volume decreases and potential energy is stored in the chamber walls 940 like a traditional spring. When the user relaxes their squeeze, the chamber walls 940 return to their original shape restoring the chamber to its original volume. The stiffness of the chamber walls is designed such that this recoil causes 30-60 Lpm to be drawn through the DPI 190.

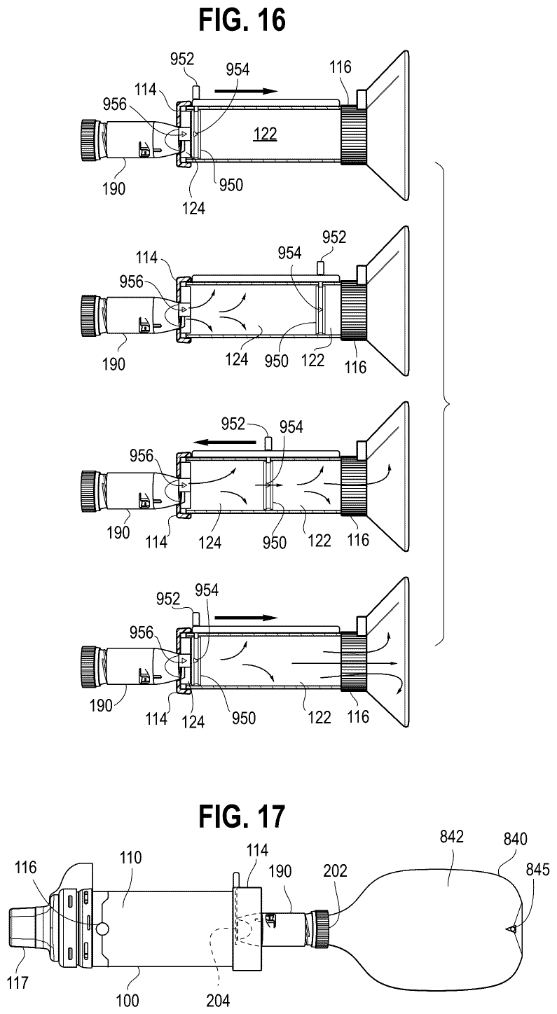

In other embodiments, shown in FIG. 16, the expander includes a movable piston 950, which separates an antechamber volume 124 from the main chamber volume 122. In this embodiment, the “main chamber volume” is defined as the volume between the inhalation valve 116 and the movable piston 950 and the “antechamber volume” is defined as the volume between the movable piston 950 and the backpiece 114 of the VHC. The piston 950 contains a one-way valve 954 which permits flow from the antechamber 124 into the main chamber 122 but not vice versa. An additional one-way valve 956 between the DPI mouthpiece and the antechamber 124 permits flow from the DPI 190 into the antechamber 124 but not vice versa.

With the DPI 190 inserted and the movable piston 950 positioned at the back of the chamber, the user slides the piston, for example using a piston handle 952 or by releasing a spring, rapidly from the back of the VHC to the front of the VHC. The movement of the piston 950 increases the antechamber 124 volume, which draws 30-60 Lpm through the DPI 190 and aerosolizes the medication into the antechamber. The user then slides the movable piston 950 to the back of the VHC which allows the suspended medication in the antechamber to pass into the main chamber 122 through the one-way valve 954. The user can then inhale through the interface 117 tidally to receive the medication. Additionally, the user or caregiver may slide the movable piston 950 forward during treatment so that dose delivery is assured.

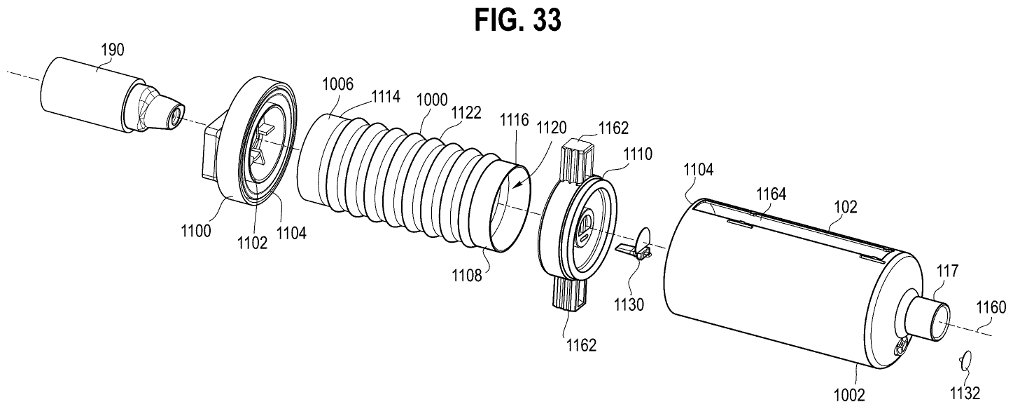

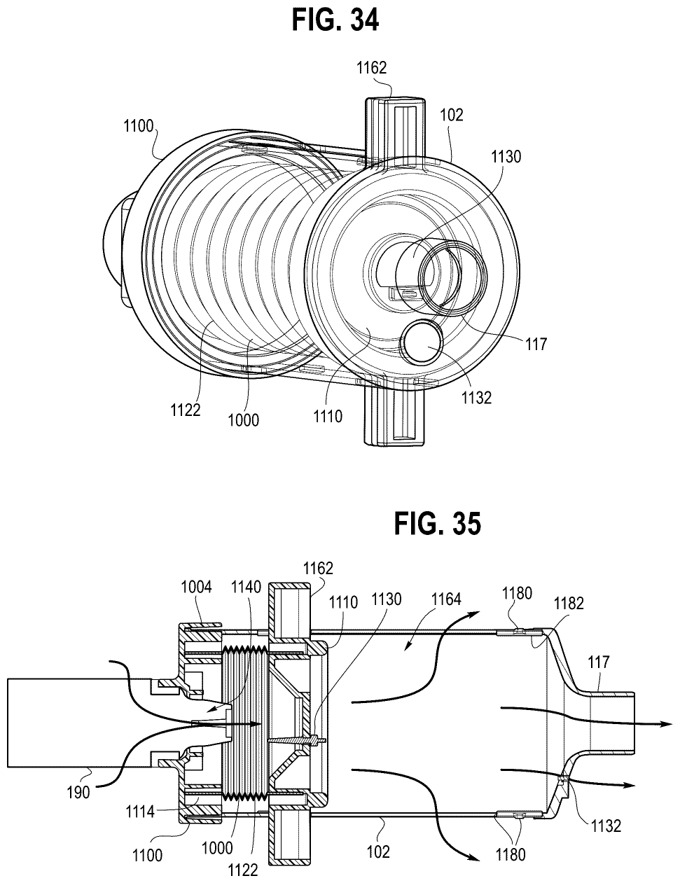

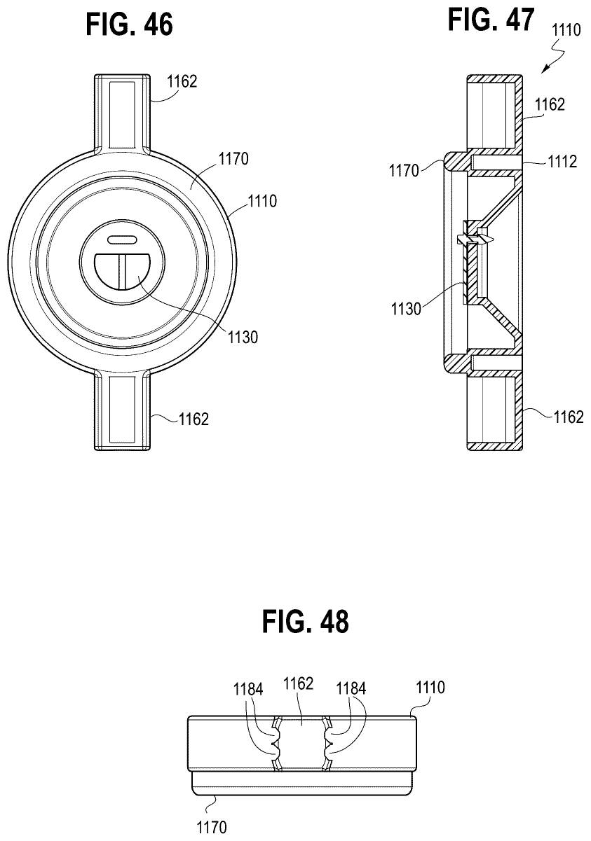

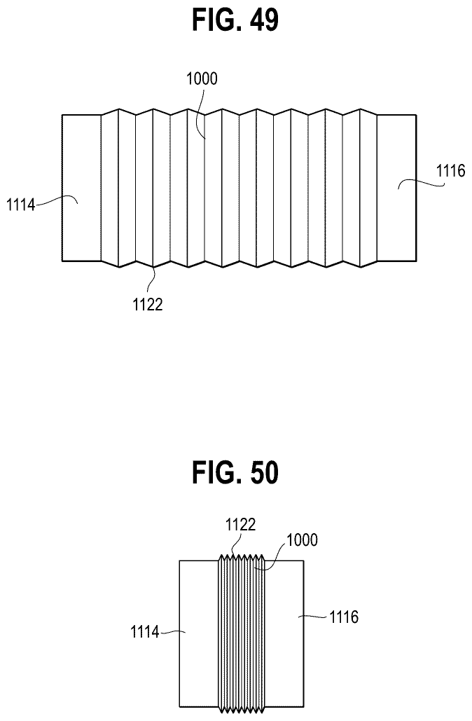

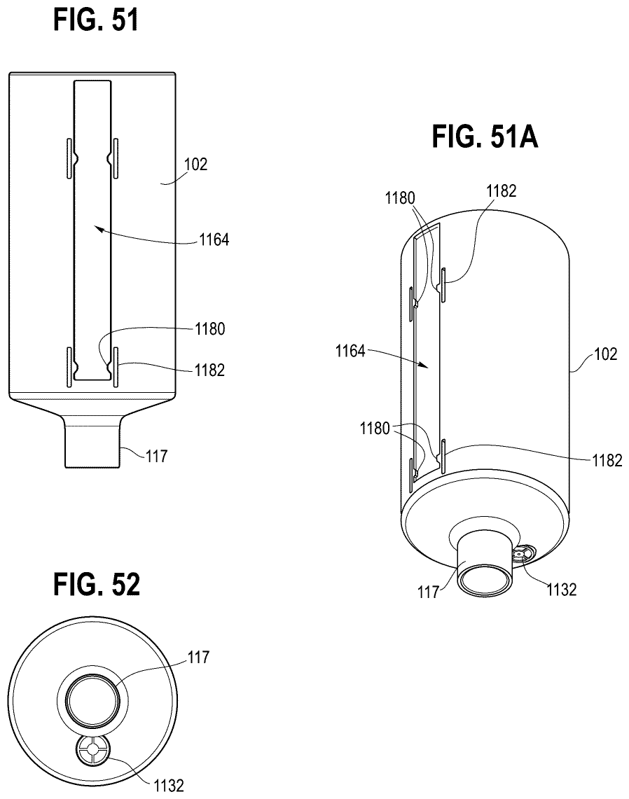

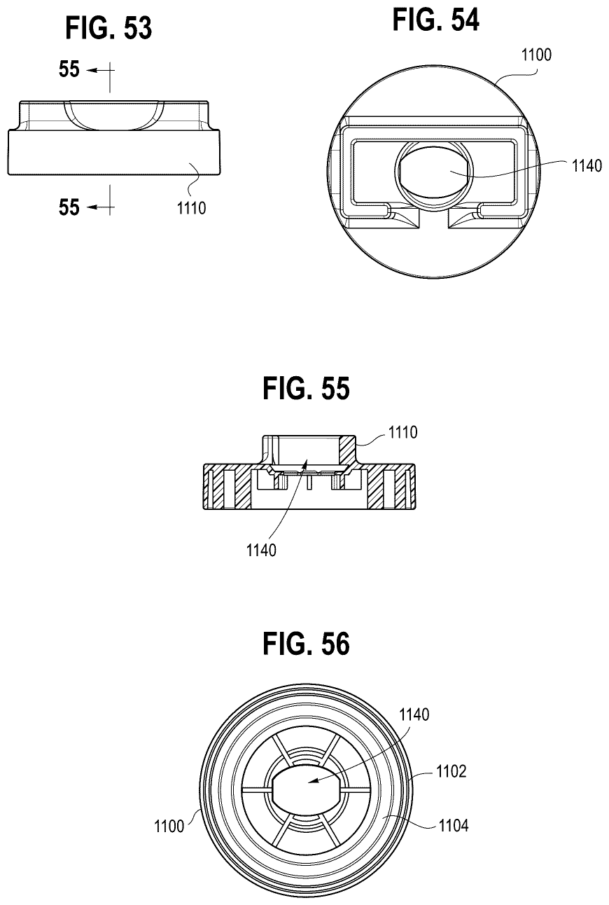

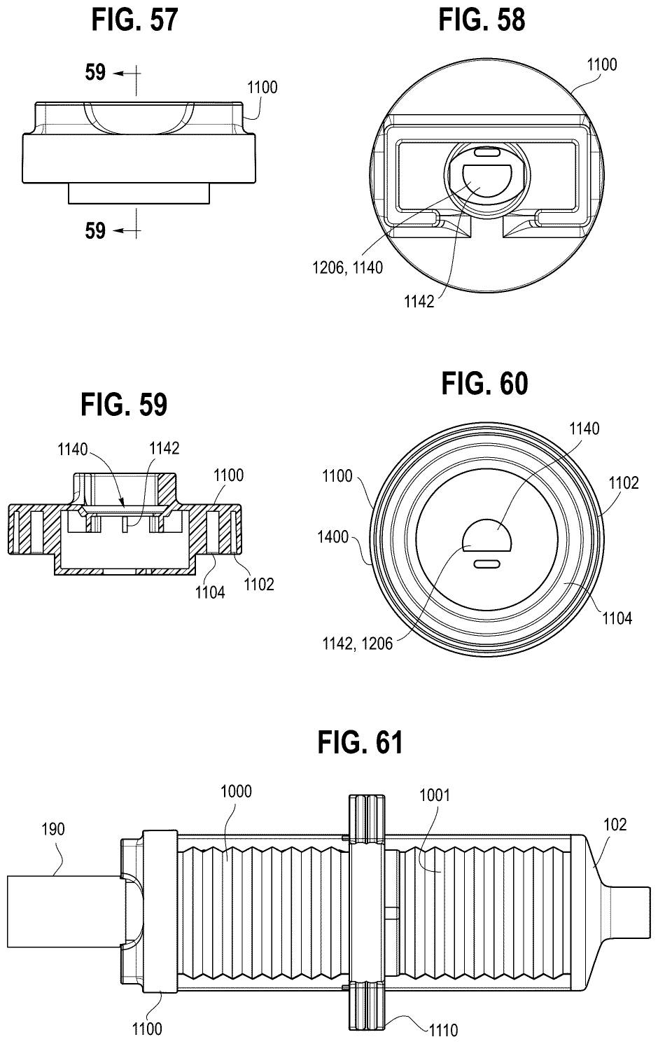

In other embodiments, shown in FIGS. 33-60, the expander also includes a collapsible wall 1000 disposed inside the chamber housing 102. The chamber housing 102 may include a cylindrical shell, which may be transparent or opaque, with a user interface 117, shown as a mouthpiece, attached to an output end 1002 of the shell and a backpiece 1100 attached to an input end 1004 of the shell. A first end 1006 of the collapsible wall 1000 is coupled to the input end 1004 of the chamber housing, and in particular to the backpiece 1100, which connects the collapsible wall 1000 and the shell. The backpiece 1100 may be configured with a pair of radially spaced annular grooves 1102, 1104, with an annular flange 1114 defining the first end 1006 of the collapsible wall inserted into the inner annular groove 1104 and the annular end of the chamber housing 102 inserted into the outer annular groove 1102. A piston 1110, otherwise referred to as an actuator, may also be configured with an annular groove 1112, with an annular flange 1116 of the collapsible wall being inserted into the groove 1112 to secure the piston to the collapsible wall. In this way, the collapsible wall 1000, together with the backpiece 1100 and piston 1110, defines an expandable interior volume 1120. The collapsible wall 1000, which has an outer diameter smaller than an inner diameter of the chamber housing 102, may be moved between a collapsed configuration, or state, and an expanded configuration, or state. In one embodiment, the collapsible wall 1000 includes circumferential pleats 1122, which may be collapsed and expanded as shown in FIGS. 35-39, 49 and 50. The output end 1002 of the holding chamber, which is positioned upstream of the piston 1110 and is in fluid communication with a one-way valve 1130 coupled to the piston, includes the user interface, such as mouthpiece 117 or mask, which may be engaged by the user 1200. A one-way exhaust valve 1132 is coupled to the output end, and is in communication with a space 1136 formed between the piston 1110 and the interior surface 1138 of the mouthpiece 117 and the ambient environment, as shown in FIG. 37, such that during exhalation, air may pass back through the mouthpiece 117 to the ambient environment, with the one-way inhalation valve 1130 preventing the exhalation air from reentering the interior volume 1120. The backpiece 1100, coupled to and connecting the input end of the chamber housing and the first end of the collapsible wall, has an opening 1140 configured to receive the dry powder inhaler 190. The backpiece 1100 may include a one-way inhalation valve 1142 in communication with the expandable interior volume 1120 defined by the collapsible wall 1000.

Referring to FIGS. 33-60, in the collapsed state, shown in FIG. 35, the piston 1110, also referred to as an actuator, is slid towards the back, or input end, of the VHC such that the space between the actuator 1110 and backpiece 1100 is minimized. It should be understood that the piston 1110 does not seal or slide against an interior surface of the holding chamber shell to define the expandable interior volume 1120, which is defined by the collapsible wall 1000, but rather simply moves inside the chamber housing 102 so as to move the collapsible wall 1000, and define one end of the interior volume. As the piston 1110, or actuator, is moved toward the input end of the holding chamber, the collapsible wall 1000 is compressed. The chamber housing 102 guides the piston and maintains an orientation thereof orthogonal to an axis 1160 of the chamber housing, while also preventing buckling of the collapsible wall 1000 since the inner diameter of the chamber housing 102 is close in size (i.e., the same or slightly larger than) the outer diameter of the collapsible wall 1000.

When the user is ready to dispense the DPI medication, the user prepares the DPI 190 for dispensing and inserts the DPI into the opening 1140 of the backpiece 1100 while the collapsible wall 1000 is in its collapsed state. The user may grasp the chamber housing 102, for example the input end thereof, with one hand, and grasp one or both of a pair of handles 1162 extending radially from the piston 1110 through slots 1164 formed in the shell at opposite 180 degree positions relative to each other. The handles 1162 may be configured as lugs, or guides, which move in the slots 1164 defining tracks. The user may grasp and rapidly slide the piston 1110 toward the front, or output end, of the chamber housing 102. The collapsible wall 1000, which is hermetically secured and sealed to the backpiece 1100 and piston 1110, draws air in exclusively through the opening 1140 in the backpiece and through the DPI 190. The one-way inhalation valve 1130 in the piston prevents air from entering the interior volume 1120 defined by the collapsible wall though the piston 1110. The speed at which the user slides the piston 1110 toward the output end, or front, of the chamber housing 102 is proportional to the flow rate of air pulled through the DPI 190 and is thereby responsible for the effective deagglomeration of the medication. During the transition of the collapsible wall 1000 from the collapsed state to the extended state, air upstream of the piston 1110 within the chamber housing 102 must displace rapidly to avoid any issues with back pressure building up and slowing the actuation. This air is free to escape the chamber housing 102 via the guide slots 1164 for the actuator, exhalation valve 1132 and/or the mouthpiece 117 at the VHC output end. The rapid actuation of the DPI 190 accelerates the medication in such a way that large particles are baffled by the piston 1110 and collapsible wall 1000. The collapsible wall 1000 and piston 1110 may be made of materials having antistatic properties, such that the fine medicament particles remain suspended within the interior volume of the collapsible wall. The user may then inhale through the mouthpiece 117 of the VHC, drawing the aerosolized medicament through the one-way valve 1130 of the piston and through the mouthpiece 117. Exhalation by the user causes an exhalation flow to move through the one-way valve on an attached mask (not shown), or through the one-way exhalation valve 1132 attached to the chamber housing. Alternately the user may exhale with their mouth removed from the output end.

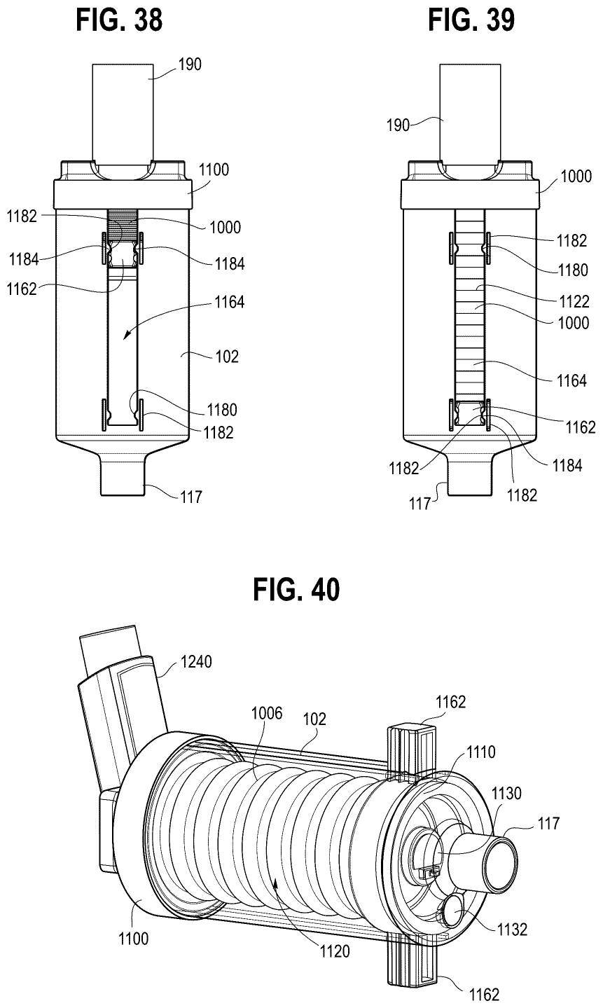

While inhaling, the piston 1110 seals against the inside surface 1138 of the output end of the chamber housing to prevent any inhalation leakage. The piston may include an annular seal member 1170, shown as a longitudinally extending protuberance or rib, which engages and seals against the interior conical surface 1138 of the chamber housing output end when the piston 1110 and collapsible wall 1000 are in the extended configuration, or state, as shown in FIG. 36. The annular seal member 1170 on the piston 1110 may be an elastic, or compressible material, such as silicone rubber or thermoplastic elastomer (TPE). Alternatively, a flexible-material seat may be formed on the conical face of the chamber housing via insert molding or as a separate component, and engage the annular seal member, which may be made of a more rigid material. The seal between the seal member 1170 and seat, or surface 1138, may be maintained via hand pressure from the user. The pressure on the seal should be maintained at a level sufficient to ensure a consistent seal is achieved. To achieve this and to provide feedback to the user as to the correct position of the actuator for inhalation, detents 1180 may be included adjacent to, or along the sides of, the chamber housing guide slots 1164 such that the detents, shown as bumps or protuberances, may be engaged with complimentary features, shown as recesses 1184 or cutouts, on the guide lugs. A slot 1182 may be formed adjacent the detent 1180, such that the detent is configured as a beam spring. In this way, the piston 1110 may be held in its extended configuration, as shown in FIG. 39. The detents 1180, and interface with recesses 1184, also hold the piston 1110 in the collapsed configuration, or state, as shown in FIG. 38. The detents 1180 also provide a visual, tactile and/or audible feedback to the user that once engaged, the piston/actuator 1110 is in either the collapsed state, ready for actuation, or in the expanded state and sealed for inhalation.

In one embodiment, the collapsible wall 1000 may be integrally formed with, or coupled to, one or more springs. The spring(s) may provide energy to expand the collapsible wall 1000 at a sufficient rate to create an appropriate flow rate through the DPI 190 to deagglomerate the drug formulation. When the spring enabled collapsible wall 1000 is fully extended, the residual force of the spring may hold the piston 1110 and seal member 1170 firmly against the mating surface 1138 on the interior of the chamber housing 102 to ensure an effective seal is achieved and maintained.

In an alternative embodiment, magnets 1190, 1192 may be disposed in or on the piston, for example on the lugs 1162, and at the output end of the chamber housing 102 such that the attraction of the magnets holds the actuator sealing ring against the inside surface of the chamber housing. A similar holding mechanism may be included at the input end of the chamber housing such that the collapsible wall 1000 is held securely in the collapsed configuration while the user loads the DPI. The magnets 1190, 1192 may also provide a similar tactile and/or audible feedback to the user when they are engaged in addition to, or as compared with, detents. Alternately, the magnets may form annular rings that, when engaged, form a hermetic seal without the need of any additional elastomeric seal by virtue of the hard and highly polished surface of the magnets.

In an alternate embodiment, shown in FIG. 61, two collapsible wall portions 1000, 1001 are utilized. The first collapsible wall 1000, as disclosed above, connects the piston and the backpiece, and a second collapsible wall 1001 connects the other side of the piston 1110 with the inside of the chamber housing 102. The second collapsible wall 1001 effectively eliminates the need of for the seal between the piston and the interior of the chamber housing when the first collapsible wall 1000 is fully extended and ready for inhalation. The second collapsible wall 1001 is hermetically sealed with the piston 1110 and the output end of the chamber housing 102. When the piston 1110 is moved fully towards the output end and ready for inhalation, the first collapsible wall 1000 is extended and the second collapsible wall 1001 is collapsed. The opposite is true when the piston 1110 is moved toward the input end. In this way, hermetically sealed interior volumes are maintained on both sides of the piston regardless of what position the piston is in, with the one way valve 1130 communicating between the interior volumes.

In one embodiment, an actuation mechanism 300 may be incorporated to release the energy stored in elastic members, for example integrated into the collapsible wall. When ready to actuate, the user actuates the release, or trigger 307, for example a button or lever, thereby releasing the detents/recesses 1180, 1184, or catches, holding the actuator at the input end, for example via the push of a button 307 which mechanically moves the detent to a point where interference no longer exists between the chamber housing detents and actuator lugs. The energy stored in the elastic members, for example one or more springs 1194, is then released, sliding/pulling the piston 1110 to the front/output end of the chamber housing 102 and actuating the DPI 190 at a sufficient rate as to deagglomerate the drug formulation as described above.

Ideal flow rates for DPIs may generally be achieved when a pressure drop of 40 cmH2O (4 kPa) is applied across the DPI. Pressure drops ranging between 10 cmH2O (1 kPa) and 40 cmH2O (4 kPa) or greater may also be acceptable. Within the ideal range of internal device volumes (i.e. 150 mL to 400 mL), the amount of work applied by the spring(s) 1194 may be calculated using the equation W=P×ΔV. In one exemplary embodiment, the volume of the collapsible wall 1000 in the expanded state is around 250 mL which balances aerosol performance and compact size of the device for usability and convenience purposes. In this embodiment, the minimum acceptable energy stored in the spring is: Wmin=Pmin×ΔVmin, or Wmin=1,000 Pa×0.000155 m3 and Wmin=0.155 J. In this embodiment, the upper range of energy storage in the spring is: Wupper=Pupper×ΔVmax, or Wupper=4,000 Pa×0.000400 m3 and Wupper=1.600 J. Therefore, in one embodiment, the spring(s) 1194 should be able to store between 0.155 J and 1.600 J of potential energy when the collapsible wall 1000 is in the compressed state. The energy stored in a linear spring 1194 is represented by E=(k×2)/2. For a preferred volume of 250 mL, the corresponding lengths of the collapsible wall (and therefore, distance of spring compression/extension) are between 11 cm and 26 cm, for a 5.2 cm and 3.5 cm internal collapsible wall diameter, respectively. The range of spring constant, k, can be obtained by setting E=0.155 J and E=1.600 J, substituting the lengths of collapsible wall in for x, and isolating for k. Doing so, we obtain spring constants in the range of 4.6 N/m to 231 N/m. Springs having other spring constants may be suitable.

Elastic energy storage integrated directly into the collapsible wall may also be achieve in various ways. In one embodiment, a compression (e.g., coil) spring may be incorporated into the walls of the collapsible wall 1000. Alternately, the elastic energy required to properly expand the collapsible wall actuation may be stored in the materials used to create the collapsible wall itself. Alternately, and as described in an earlier embodiment, two collapsible walls 1000, 1001 may be used, with each wall having an integrated spring(s). The first collapsible wall 1000 contains a compression spring which is configured to push the piston 1110 from the input end to the output end during actuation. The second collapsible wall 1001 is configured with a second spring, which may be a tension spring, that pulls the piston 1110 from the input end towards the output end during actuation. The advantage of this approach divides the amount of force to be applied between the two springs which may allow for smaller form factor or better dynamics during actuation. This combines with the earlier advantages described of using two collapsible walls for sealing purposes.

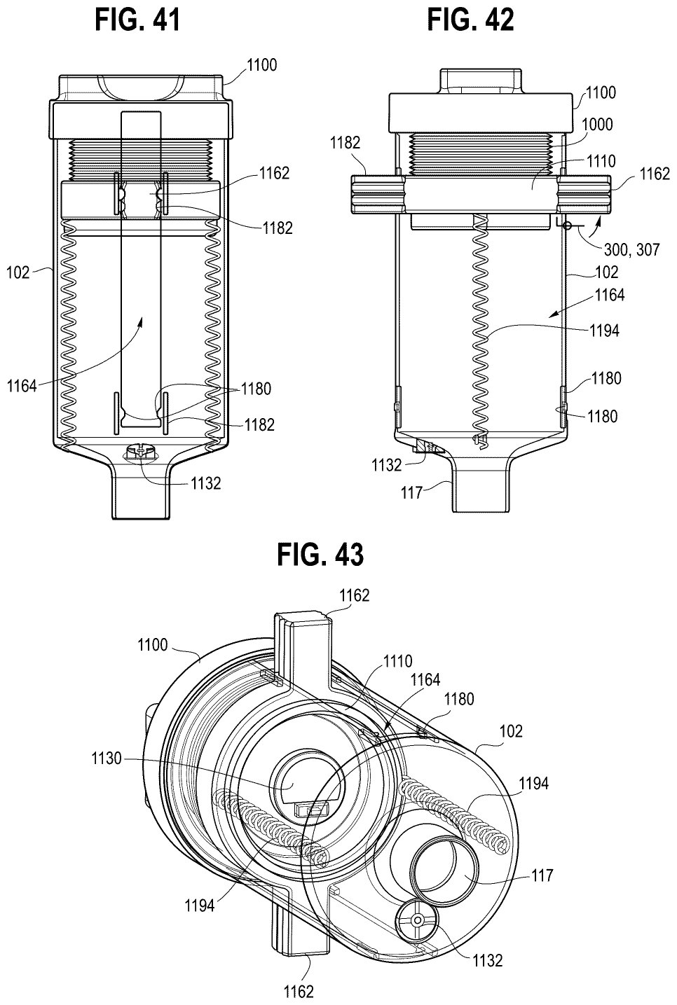

Alternatively, tensile springs may be incorporated into the design and serve as the energy storage mechanism as shown in FIGS. 41-43. In one embodiment, a pair of springs 1194 may be arranged within, or interiorly in, the chamber housing 102 so as to minimize any pinch points and contain any dislodged parts. The springs 1194 are fastened to the chamber housing 102 and to the piston 1110 radially outwardly from the annular seal member 1170 on the piston 1110, which avoids any need for a pass-through opening and the attendant possibility of inhalation leak paths during normal use. An alternative to linear tensile or compression springs is a single constant force spring designed to apply a constant force over the entire travel of the actuator.

As shown in FIG. 40, the valved holding chamber 100, with the collapsible wall 1000 secured (e.g., with the detent) in the expanded configuration, and sealed against the chamber housing 102, may be used as a conventional holding chamber, with a pressurized metered dose inhaler (PMDI) 1240 inserted into the opening 1140 of the backpiece 1100. The PMDI 1240 may be actuated to dispense a dose of medicament into the interior volume 1120 of the expanded interior volume, with the aerosolized medication thereafter being drawn out of the holding chamber through the one-way valve 1130 during inhalation. In this way, one or ordinary skill in the art should understand that the valved holding chamber 100 may be connected with a variety of medicament delivery devices, including for example and without limitation the PMDI and/or the DPI.

### Positive Pressure

In one embodiment, shown in FIG. 17, a positive pressure source 840, configured as a resilient, hollow member, or bladder 842, is attached to the inlet 202 of a DPI 190 such that the air inlets 202 of the DPI are exposed to the internal volume of the bladder 842 and the mouthpiece, or outlet 204, of the DPI is exposed to atmosphere and free to interface with the VHC backpiece. The attachment of the positive pressure source 840 to the DPI provides an airtight seal. Attached to the bladder is a low-resistance one-way valve 845 that permits air to flow into the bladder from atmosphere but not vice versa. With the DPI attached to the VHC and the bladder attached to the DPI, the user squeezes the bladder 842 to decrease its volume. The air within the bladder, or positive pressure source, is driven through the inlet(s) 202, through the DPI and into the VHC 100, aerosolizing the medication in the process. The suspended drug is then inhaled from the VHC tidally.

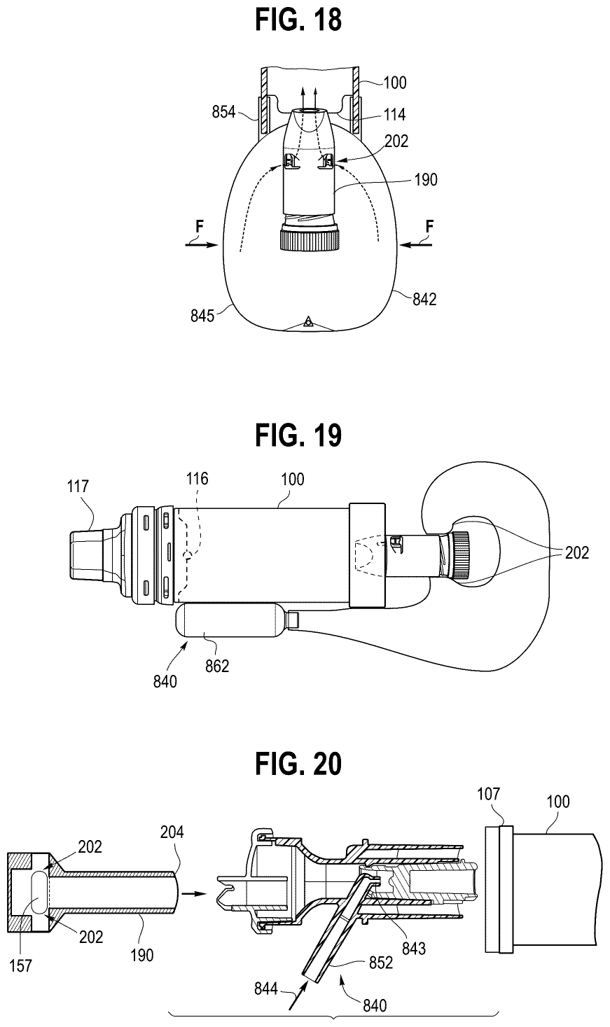

In another embodiment, shown in FIG. 18, the bladder 842 contains a mounting portion 854 that is coupled to the chamber housing 102, with the DPI 190 disposed in the bladder and coupled to the backpiece 114. The bladder 842 may be squeezed or compressed so as to create a positive pressure that flows through the DPI 190 and into the VHC 100.

In another embodiment shown in FIG. 19, the positive pressure source 840 may include a compressed gas source 862, which is coupled to the air inlets 202 of the DPI and supplies a positive pressure or forced air through the DPI in a similar fashion as described above.

In the embodiment shown in FIG. 20, a positive air pressure device 840 is coupled between the DPI and the VHC. The device supplies a compressed-air 844 which is directed into the input end 107 of the VHC. The compressed air creates a vacuum at the outlet 204 of the DPI. When the user triggers the compressed air to flow through a port 852, the airflow is accelerated through a nozzle 843 and allowed to expand within the vacuum source device, ultimately exiting through the VHC 100. This fast-moving, expanding air causes air to be entrained through the DPI 190 at a rate of 30-60 Lpm until the compressed air source 844 is turned off. The aerosolized medication then remains suspended in the VHC for tidal inhalation.

### DPI and VHC Interface

The coupling and fluid communication between the DPI 190 and VHC 100, and minimizing of fluid leaks therebetween, may improve the overall performance of the system. The interface between the DPI and VHC may be located at the backpiece 114, or at other locations, such that the flow into the VHC is parallel to the axis of the chamber, or transverse thereto, for example orthogonal or at some other angle. It should be understood that the same interface, or DPI couplers, may also be used between the DPI and the positive pressure source.

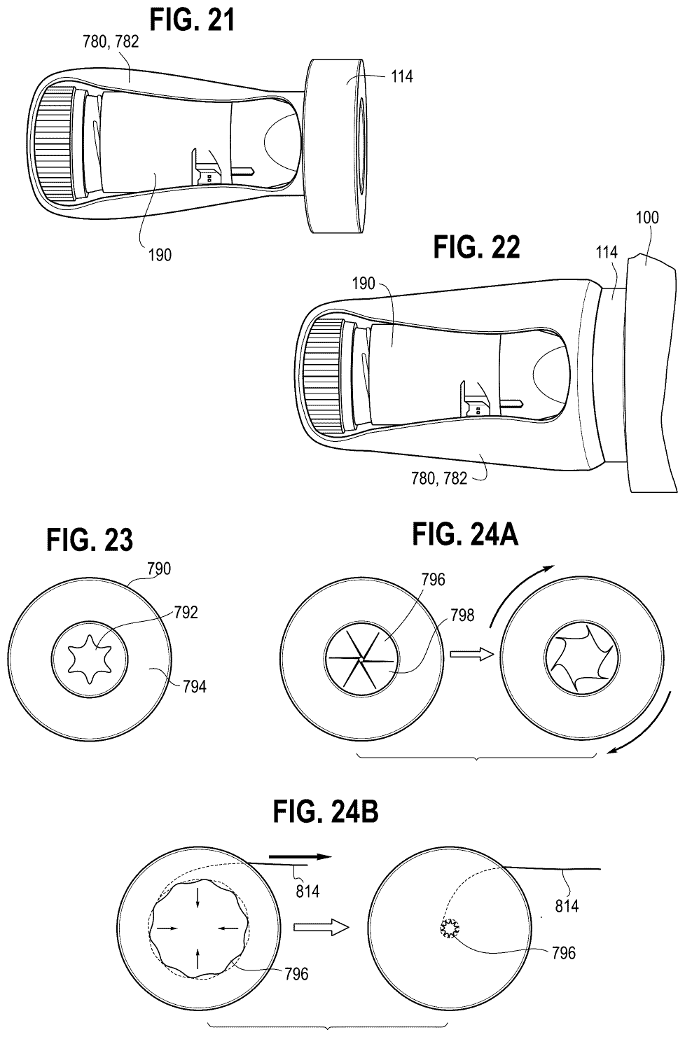

In one embodiment, a DPI coupler 780 is configured as an elastic band 782, as shown in FIGS. 21 and 22, with the band 782 wrapping around the backside of the DPI and biasing the mouthpiece into engagement with the backpiece 114. The elastic band 782 may be molded into the VHC backpiece component 114. The band 782 may be secured to an interior portion of the backpiece 114 as shown in FIG. 21, or an outer portion thereof as shown in FIG. 22.

In one embodiment, molded silicone or thermoplastic elastomer (TPE) backpieces may be designed and manufactured specifically for each DPI on the market. In other embodiments, the backpiece may be designed such that one backpiece serves to fit a subset of DPIs while other backpiece(s) serve to fit other subset(s) of DPI.

In an alternate embodiment, the VHC backpiece is molded from a plastic with softening temperature (“transition temperature”) below ˜100 degrees C. (temperature of boiling water, depending on altitude) and greater than 50 degrees C. When the user purchases the VHC, the use may heat the backpiece to the transition temperature and the specific DPI of the user will be inserted into the backpiece such that the backpiece conforms to the DPI mouthpiece. When cooled, the backpiece will remain in its new shape which conforms to the user's DPI mouthpiece while still maintaining its interface with the VHC body. Once this “molding” occurs, the backpiece will be unsuitable for use with DPIs other than the type used in the molding process. This material and process are like those in custom mouthguard products on the market.

In other embodiments, a universal DPI coupler 790 may be used. For example, in the embodiment of FIG. 23, the backpiece is made of a very soft and malleable material capable of significant reversible deformation to conform to the range of DPI mouthpiece shapes. For example, the backpiece may be made of air-filled silicone. The deformable material may be a localized region 792 adjacent to and surrounding the DPI port, with a firmer material defining a region 794 positioned radially outwardly therefrom and maintaining a secure connection at the interface between the backpiece and the VHC body.

Referring to FIGS. 24A and B, the size of the DPI port 796 may be varied. For example, as shown in FIG. 24A, the size of the opening may be increased by applying a torque, or twisting action to the backpiece 114, which radially retracts interface segments 798, allowing the user to insert the DPI more easily. Once the torque is removed, the segments 798 are biased toward the closed position, conforming around the DPI mouthpiece in the process. Likewise, in the embodiment of FIG. 24B, a drawstring 814 may be used to increase and decrease the size of the DPI port 796 to secure the DPI.

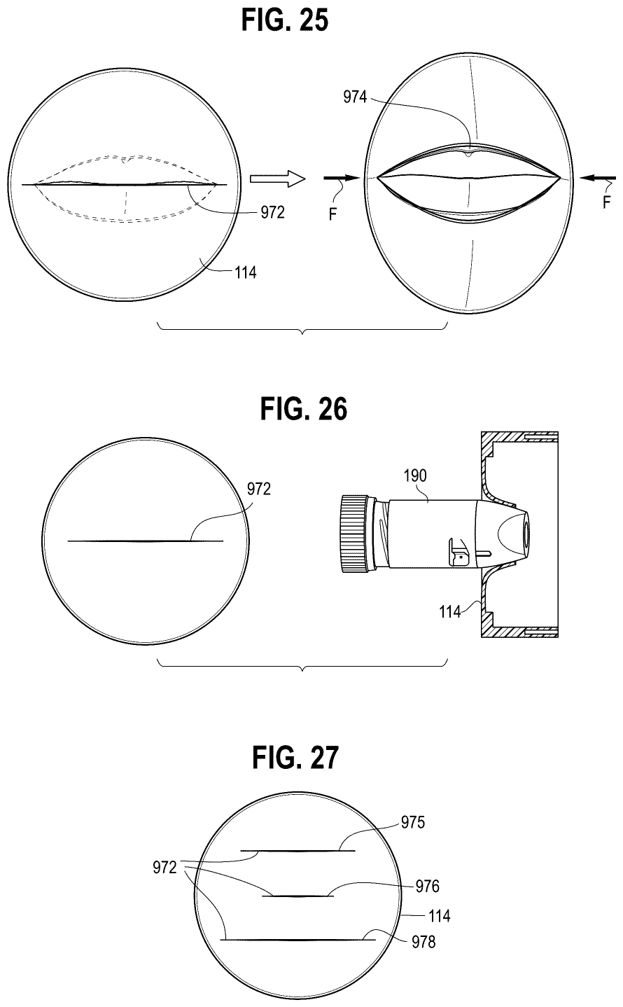

Referring to FIG. 25, one embodiment of the DPI port is configured as a simple slit 972 in the backpiece. When the user applies a compressive force (F) along the axis of the slit 972, the material above and below the slit buckles outward providing an opening 974 for the DPI to be inserted. Once the force (F) is removed, the backpiece 114 returns, or is biased, to its original state, conforming around the DPI mouthpiece in the process.

Referring to FIG. 26, the slit 972 may be designed such that insertion of the DPI easily opens the slit and retains the DPI without any additional forces or torques being applied by the user. The VHC backpiece may also contain multiple slits 975, 976, 978, for example having different lengths, which are better suited to fit the range of DPIs on the market as shown in FIG. 27.

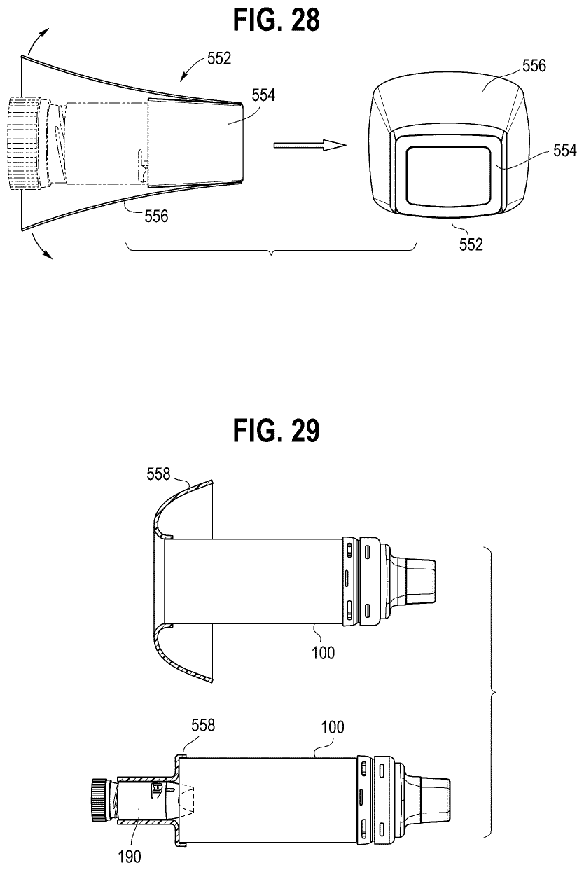

Referring to FIG. 28, an adapter 552 may be configured with one section 554 made of rigid plastic in the shape of a common MDI mouthpiece which easily and effectively interfaces with the current MDI adapter (i.e. “backpiece”) of the VHC. A second section 556 of the adapter may interface with the various DPI mouthpieces and stay permanently attached. This attachment mechanism between the adapter and DPI mouthpiece may rely on frictional forces between the DPI mouthpiece and silicone flaps of the adapter or may be aided through adhesives. The final assembly is a DPI with an attached mouthpiece resembling that of an MDI.

Referring to FIG. 29, the backpiece includes a resilient tube 558, made for example of silicone. When inserting the DPI, the walls of the tube 558 are folded or rolled up over themselves, the DPI is inserted and the walls of the tube are unfolded/unrolled back over the DPI. Since the diameter of the DPI mouthpiece is larger than the resting diameter of the tube, the compressive force of the tube on the DPI secures it in place.

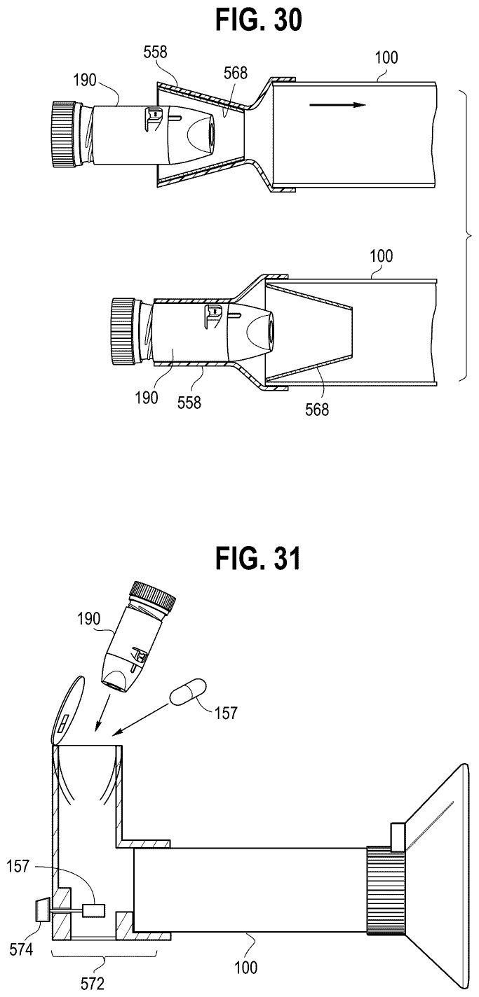

In a similar embodiment, shown in FIG. 30, a rigid expander 568 may be used to temporarily stretch the tube 558 larger so the DPI can fit inside. The expander 568 is then slid out of the way to permit the silicone tube to recoil and secure the DPI. This expander may be a part of the VHC backpiece or a separate component from the VHC.

In one embodiment, shown in FIG. 31, the DPI medication may be removed from the DPI and be loaded into an integrated VHC DPI/backpiece 572. For capsule based DPIs, the capsules 157 would be loaded directly into this VHC backpiece instead of their respective DPI. The custom VHC backpiece contains a button-activated puncture mechanism 574 for any capsules that are inserted. Once the drug is loaded into this backpiece, the user generates the required flows to deagglomerate and aerosolize the drug, with or without the aid of the actuation mechanisms as disclosed herein.

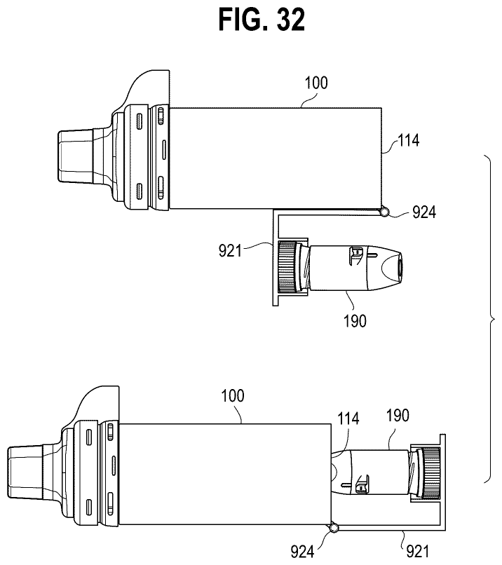

In another embodiment, shown in FIGS. 14A, 14B and 32, the DPI may be secured to the VHC using a hinged carrier 921, which may be folded against the VHC 100 into a compact device when not in use. When ready to use, the unfolding of the carrier 921, for example by pivoting of the carrier 921, automatically inserts the DPI mouthpiece into the VHC body through an airtight interface. Optionally, this unfolding mechanism can also serve to increase chamber volume from a collapsed state to an expanded state, thereby priming the system for actuation using a push-button trigger and movable valve, as discussed above with respect to FIGS. 14A and B.

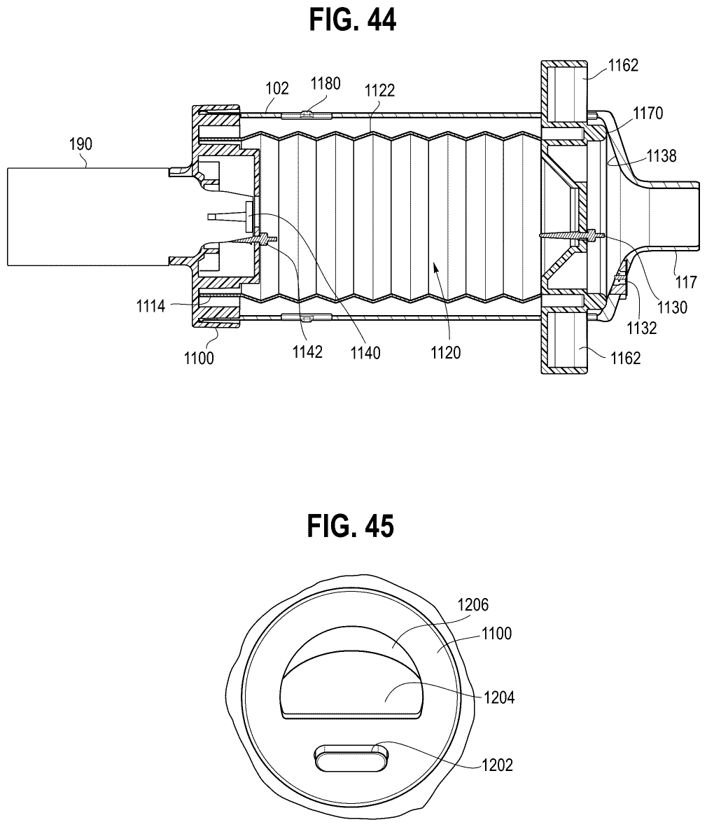

Referring to FIGS. 44, 45 and 53-60, the user may want to take more than one dose of DPI medication and may not want to remove the DPI 190 from the backpiece 1100 to reload the medication. With the collapsible wall 1000 in the extended state, if the user were to slide the piston 1110 to the collapsed state, some of the air in the interior volume 1120 defined by the collapsible wall 1000 may be forced out and through the opening 1140 and the DPI 190 (back through the DPI mouthpiece), carrying any loaded medication with the back flow in the event the user takes this step after reloading the DPI. In such a situation, the user may receive a reduced amount of medication during the subsequent actuation and inhalation. To prevent such backflow from occurring, the one-way valve 1142 may be incorporated into the backpiece 1100. During collapse of the collapsible wall 1000, pressure inside of the interior volume 1120 rises and is driven through the inhalation valve 1130. The pressure forces the one-way valve 1142 in the backpiece 1100 to remain closed, preventing flow from passing back through the DPI 190. During actuation, when the piston 1110 slides to the output end and is read for inhalation, the one-way valve 1142 may open, e.g., by deflection of a flap 1204 or petal, be positioned out of the drug flow path to avoid excessive impaction from the drug colliding with it, as shown for example FIG. 45 where 60 L/min flow is being pulled through the valve 1142. The attachment point 1202 of the one-way valve is located adjacent an edge of the valve 1142, and below the opening 1206, such that the attachment point 1202 is positioned off-center to the DPI's drug exit path thereby providing a maximum opening for drug flow without impaction on the valve.

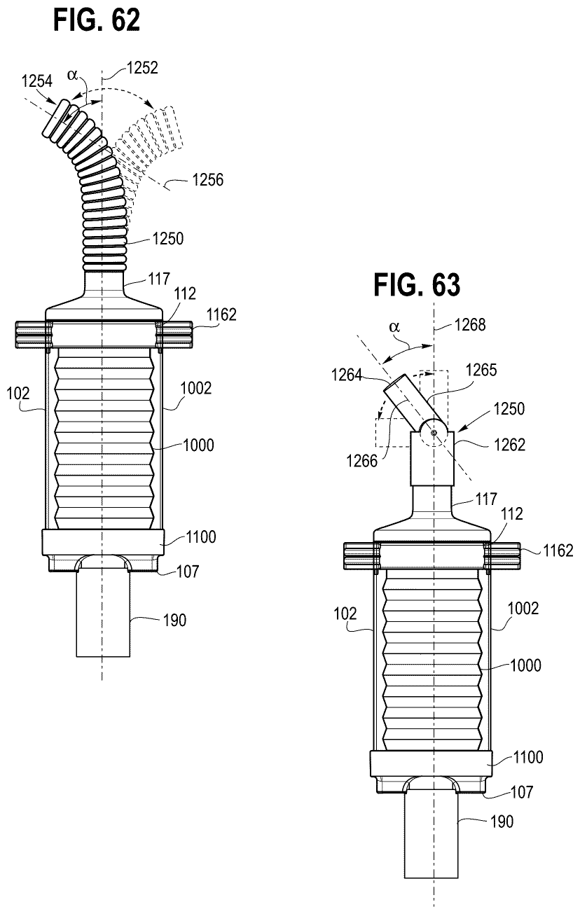

Referring to FIGS. 62-69, it may be preferred by the manufacturer of the DPI that the orientation thereof be maintained in a certain orientation during medication loading and administration. For this reason, it may be desirable to have the DPI-VHC be able to function in both the horizontal and vertical orientations, or other orientations therebetween. When horizontal, the mouthpiece or outlet described in the above embodiments is in a comfortable position for the user to inhale from. The same can be said if a mask is attached to the mouthpiece or outlet. However, when the DPI-VHC is held vertically, or at an angle relative to the horizontal orientation, it may be awkward or uncomfortable for the user to lean over the DPI-VHC to achieve a good seal on the mouthpiece or optionally attached mask or other user interface. In this situation, it may be beneficial to have an optional angled attachment that could connect the mouthpiece or output end to the user's mouth.

FIGS. 62-69 illustrate various a user interface adapter 1250, 1260, 1270, 1280, which may be coupled to, or define, the output end 112, 1002 of the chamber housing 102. The chamber housing defines or has a longitudinal axis 1252 extending between the input end 107 and the output end 112, 1002. The longitudinal axis 1252 may have a horizontal or vertical orientation, or some orientation therebetween, depending on the device and position of the user. The user interface adapter includes or defines an outlet opening 1254, 1264, 1274, 1284 defined by a second longitudinal axis 1256, 1266, 1276, 1286, wherein the second longitudinal axis define an acute angle (α) therebetween, and are non-parallel the user interface is in a use position, for example an orientation of α=45 or 90 degrees depending on the orientation of the holding chamber and user. As shown in FIG. 62, the user interface adapter 1250 may include a flexible tube defining the orientation of the opening, wherein the flexible tube is reconfigurable between a plurality of use positions, wherein the first and second longitudinal axes define acute angles relative to each other. In one embodiment, the flexible tube is configured as a corrugated tube mouthpiece extension, that may be oriented at an angle between and including 0° and 90°, while also being bendable or flexible at any orientation revolving 360 degrees around the axis 1252. The user interface adapter 1250, 1260, 1280 may be removably connected to the mouthpiece 117, for example with a simply press fit.

Referring to FIGS. 62 and 63, user interface adapter 1260 includes a first tube portion 1262 coupled to the chamber housing 102, for example by being disposed on the mouthpiece 117, and extending along the first longitudinal axis 1268, and a second tube portion 1265 pivotally attached to the first tube portion 1262, for example about a pivot or hinge defining a pivot axis. The swivel joint between the tube portions 1262, 1265 allows the opening 1264, and axis 1256 thereof, to be oriented relative to the axis 1252 of the holding chamber.

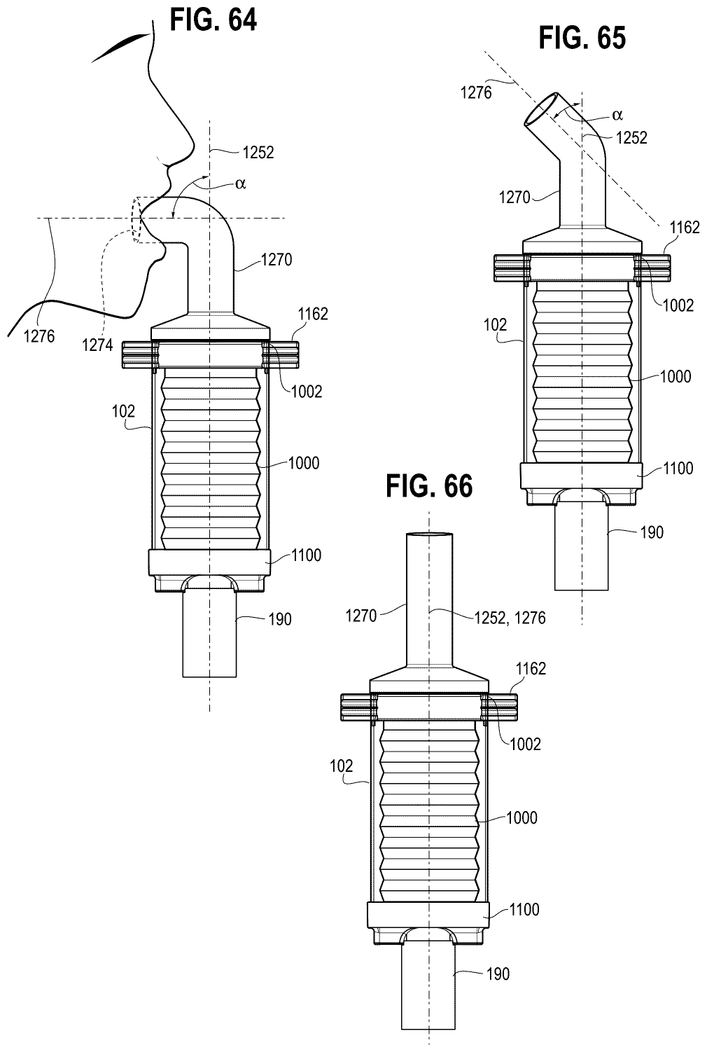

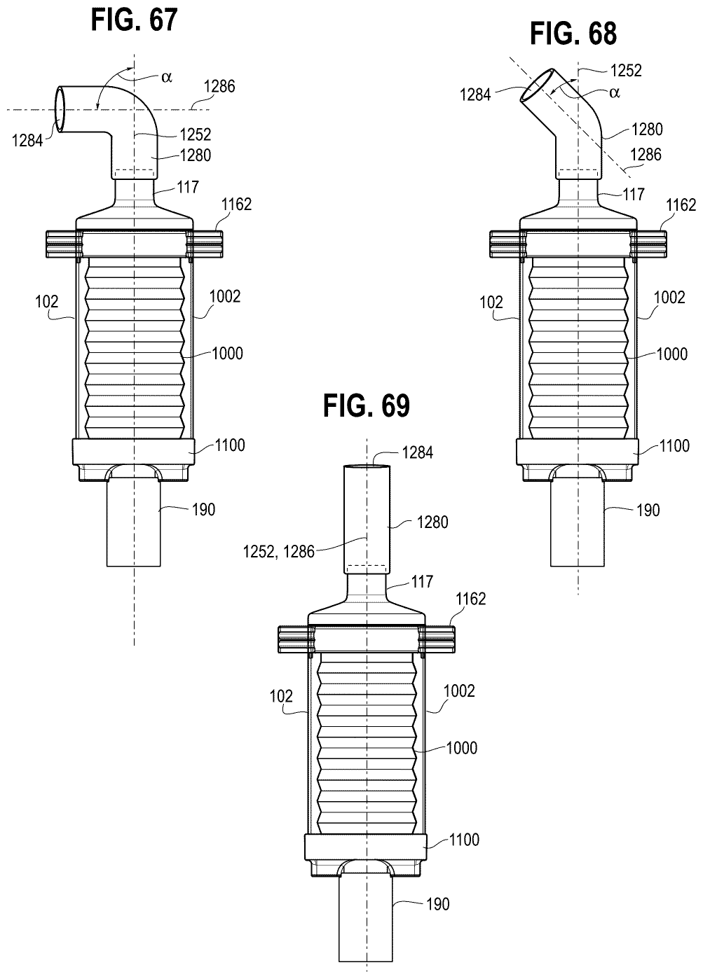

Referring to FIGS. 64, 69, the user interface adapter 1270, 1280 may be configured to provide a fixed-angle for the opening 1274, 1284 of the mouthpiece extensions, and axes 1276, 1286 thereof, ranging from 0° to 90° including for example 90 degrees (FIGS. 64, 67), 45 degrees (FIGS. 65, 68) and 0 degrees (FIGS. 66, 69). The user interface adapter 1270 may be connected to the mouthpiece 117 in a removeable or fixed manner, as shown in FIGS. 67-69, or be integrally formed and connected to the end of the holding chamber shell so as to define the interior volume. It should be understood that any number of possible angles (α) may be defined.

Angles ranging from 0 degrees to 90 degrees are preferred. The adapter may serve as both a mouthpiece and mask adapter for any optional mask attachments. In various embodiments, the angled adapter and flexible tube may be molded into the chamber housing such that it integral to it.

It should be understood that the user interface adapters shown in FIGS. 62-69 may be used with holding chambers, including valved holding chambers, besides those show in FIGS. 62-69, including without limitation holding chambers not having collapsible walls disposed therein. Rather, the holding chambers simply include chamber housings having an input end, an output end and an interior volume, with the user interface adapter disposed at the output end so as to allow the user to interface with the holding chamber in orientations other than a horizontal orientation.

Although the present invention has been described with reference to preferred embodiments, those skilled in the art will recognize that changes may be made in form and detail without departing from the spirit and scope of the invention. As such, it is intended that the foregoing detailed description be regarded as illustrative rather than limiting and that it is the appended claims, including all equivalents thereof, which are intended to define the scope of the invention.

1. A valved holding chamber comprising:
a holding chamber adapted to contain a substance and comprising an input end adapted to receive a dry powder inhaler, an output end and an interior volume defined between the input end and output end;
an inhalation valve disposed at the output end of the holding chamber and moveable to an open position in response to an inhalation flow through the interior space; and
a vacuum generator in fluid communication with the interior space, wherein the vacuum generator is configured to create a vacuum in the interior space.

2. The valved holding chamber of claim 1 wherein the interior volume comprises a main chamber separated from an ante chamber by a release valve, wherein the vacuum generator is in fluid communication with the main chamber such that the vacuum is created in the main chamber, and wherein the ante chamber is in fluid communication with the input end.

3. The valved holding chamber of claim 2 wherein the vacuum generator comprises a retractable piston.

4. The valved holding chamber of claim 3 wherein the retractable piston is disposed in the main chamber.

5. The valved holding chamber of claim 3 wherein the retractable piston is disposed in an auxiliary chamber in fluid communication with the main chamber.

6. The valved holding chamber of claim 2 wherein the release valve is coupled to an actuator, wherein the release valve is moveable in response to an input from the actuator from a closed position, wherein the release valve separates the main chamber and the ante chamber, and an open position, wherein the main chamber is in fluid communication with the ante chamber.

7. The valved holding chamber of claim 2 wherein the vacuum generator comprises a pump.

8. The valved holding chamber of claim 7 wherein the pump comprises a manual pump.

9. The valved holding chamber of claim 7 wherein the pump comprises an electric pump.

10. The valved holding chamber of claim 2 wherein the vacuum in the main chamber comprises a minimum of −10 cmH2O to −40 cmH2O.

11. The valved holding chamber of claim 11 wherein the vacuum in the main chamber is less than or equal to −821 cmH2O.

12. The valved holding chamber of claim 2 further comprising a flow resistor disposed between the input end and the main chamber.

13. The valved holding chamber of claim 2 further comprising at least one auxiliary air inlet in fluid communication with the main chamber.

14. A valved holding chamber comprising:
a holding chamber adapted to contain a substance and comprising an input end adapted to receive a dry powder inhaler, an output end and an expandable interior volume defined between the input end and output end;
an inhalation valve disposed at the output end of the holding chamber and moveable to an open position in response to an inhalation flow through the interior volume; and
an expander coupled to the holding chamber, wherein the expander is moveable from a first configuration, wherein the expandable interior volume comprises a first interior volume, and a second configuration, wherein the expandable interior volume comprises a second interior volume, wherein the second interior volume is greater than the first interior volume.

15. The valved holding chamber of claim 14 wherein the expander comprises a piston disposed in the interior volume.

16. The valved holding chamber of claim 15 further comprising a spring biasing the piston from a first position, wherein the piston defines in part the first interior volume, to a second position, wherein the piston defines in part the second interior volume.

17. The valved holding chamber of claim 16 further comprising an actuator coupled to the piston and/or spring, wherein the actuator is configured to release the spring so as to bias the piston from the first position to the second position.

18. The valved holding chamber of claim 15 wherein the piston is disposed in the main chamber.

19. The valved holding chamber of claim 18 wherein the piston comprises a one-way valve.

20. The valved holding chamber of claim 19 wherein the one-way valve comprises a first one-way valve, and further comprising a second one-way valve disposed at the input end.

21. The valved holding chamber of claim 15 wherein the retractable piston is disposed in an auxiliary chamber in fluid communication with the main chamber.

22. The valved holding chamber of claim 15 wherein the retractable piston comprises a pull handle configured to be grippable by a user.

23. The valved holding chamber of claim 14 wherein the expander comprises a collapsible wall defining in part the holding chamber.

24. The valved holding chamber of claim 23 wherein the collapsible wall comprises telescoping sections.

25. The valved holding chamber of claim 23 wherein the collapsible wall comprises pleats.

26. The valved holding chamber of claim 23 wherein the collapsible wall comprises a deformable elastic side wall defining the holding chamber.

27. The valved holding chamber of claim 18 wherein the expander further comprises a collapsible wall disposed inside the chamber housing, wherein a first end of the collapsible wall is coupled to the input end of the chamber housing and a second end of the collapsible wall is coupled to the piston, wherein the collapsible wall defines the expandable interior volume.

28. The valved holding chamber of claim 27 further comprising a backpiece coupled to and connecting the input end of the chamber housing and the first end of the collapsible wall, wherein the backpiece comprises an opening configured to receive the dry powder inhaler.

29. The valved holding chamber of claim 28 wherein the backpiece comprises a one-way inhalation valve in communication with the expandable interior volume defined by the collapsible wall.

30. The valved holding chamber of claim 27 wherein the output end of the holding chamber comprises a one-way exhaust valve positioned upstream of the piston.

31-45. (canceled)
